(19) United States
(12) Patent Application Publication
(10) Pub. No.: US 2026/0308668 A1
(43) Pub. Date: Oct. 8, 2026

(54) Methods and Systems for Sensitive and Multiplexed Analysis of Short RNA Sequences

(71) Applicant: Jia GUO, Chandler, AZ (US)

(72) Inventor: Jia GUO, Chandler, AZ (US)

(73) Assignee: Arizona Board of Regents on Behalf of Arizona State University, Scottsdale, AZ (US)

(21) Appl. No.: 19/641,382

(22) Filed: Apr. 7, 2026

Related U.S. Application Data
(60) Provisional application No. 63/784,821, filed on Apr. 7, 2025.

(51) Int. Cl. C12Q 1/6841; C07D 311/82; C07D 495/04

(52) U.S. Cl. CPC C12Q 1/6841; C07D 311/82; C07D 495/04

(57) ABSTRACT

Methods for multiplexed single-cell spatial nucleic acid profiling in intact cells are described herein. The methods enhance multiplexed nucleic acid staining specificity and imaging sensitivity by several orders of magnitude. Also provided are novel probes and compounds for use in the methods.

### CROSS-REFERENCE TO RELATED APPLICATIONS

This application claims benefit of priority to U.S. Provisional Patent Application No. 63/784,821, filed Apr. 7, 2025, the content of which is incorporated by reference in its entirety.

### STATEMENT REGARDING FEDERALLY SPONSORED RESEARCH

This invention was made with government support under R01 GM127633 awarded by the National Institutes of Health. The government has certain rights in the invention.

### BACKGROUND

Advances in single-cell and spatially resolved transcriptomics have revolutionized biological research by revealing cellular heterogeneity while preserving spatial context, which is essential for understanding tissue architecture, cellular interactions, and disease mechanisms. Next-generation sequencing (NGS) and microarray technologies have enabled comprehensive whole genome transcriptomics analyses, but these methods require transcript isolation and purification, resulting in the loss of spatial information of transcripts within the tissue. On the other hand, imaging-based RNA profiling methods, such as molecular beacons, and single-molecule fluorescent in situ hybridization (smFISH), preserve spatial context, and provide single-cell resolution. However, the number of transcripts detected using these approaches is limited by spectral overlap and the availability of labeled fluorophores, constraining their multiplexing capacity. To overcome these limitations, multiplexed in situ RNA analysis techniques have emerged, including reiterative hybridization, multiplexed error-robust FISH (MERFISH), sequential hybridization, and in situ sequencing (ISS). Despite their advancements, these methods often lack sufficient signal amplification, leading to low sensitivity, particularly in formalin-fixed paraffin-embedded (FFPE) tissue—a widely used specimen in clinical diagnostics and disease research.

Additionally, the huge number of varied RNA isoforms and short RNAs, such as microRNA, non-coding RNA and circular RNA, play critical roles in normal physiology and disease pathogenesis. Multiplexed single-cell spatial short RNA profiling in intact tissues holds great promise to unlock major mysteries in biology and medicine. This approach can reveal the gene expression regulation, spatial organization, and interactions of the diverse cell types in many multicellular organisms, such as brain tissues, solid tumors and developing embryos. However, it is extremely challenging to study short RNAs in highly autofluorescent formalin-fixed paraffin-embedded (FFPE) tissues, which are the most abundant archived specimens in clinical tissue banks. With the sensitivity of <0.25 transcript count per gene per cell, the current multiplexed RNA imaging methods are unable to profile short RNAs in FFPE tissues

This highlights the critical need for highly multiplexed RNA profiling techniques that can also profile short RNAs and are compatible with FFPE tissues, which can improve diagnostic accuracy and deepen our understanding of disease pathogenesis.

### SUMMARY

In an aspect, provided herein is a method for multiplexed in situ analysis of nucleic acids in a tissue, the method comprising: a) contacting the tissue with a target probe or a pair of target probes capable of hybridizing to a target nucleic acid; b) contacting the tissue with a signal amplification molecule capable of binding to the target probe or pair of target probes, wherein the signal amplification molecule comprises a plurality of horseradish peroxidase (HRP) molecules; c) contacting the tissue with a cleavable hapten tyramide (CHT) in a tyramide signal amplification (TSA) buffer; d) contacting the tissue with HRP-conjugated streptavidin (SA-HRP); e) contacting the tissue with a cleavable fluorophore tyramide (CFT) in a TSA buffer; f) imaging the tissue; g) contacting the tissue with an HRP deactivating agent; h) contacting the tissue with biotin; i) repeating steps a) through h) for a plurality of rounds, wherein in each round, the target probe is capable of hybridizing to a different target nucleic acid; and wherein the fluorophore of each CFT is different than an immediately previous round.

The method may further comprise j) contacting the tissue with a first cleavage solution comprising tris(2-carboxyethyl)phosphine (TCEP); k) contacting the tissue with a second cleavage solution comprising 1,3,5-Triaza-7-phosphaadamantane (PTA); 1) contacting the tissue with DNase I; m) contacting the tissue with a DNase I quenching buffer; n) contacting the tissue with 70% formamide; and o) repeating steps a) through n).

In embodiments, step b) is done for about 30 minutes at about 40° C. In embodiments, step c) is done for about 30 minutes at about 40° C. In embodiments, between steps c) and d) the method may further comprise washing the tissue in a buffer for about 2 minutes at about room temperature. In embodiments, step d) is done for about 30 minutes at about room temperature. In embodiments, b steps d) and e) are done in an incubation buffer comprising 2×SSC, 1 mg/mL BSA, and 0.1% Tween-20.

The target nucleic acid may be an mRNA. The target nucleic acid may comprises a exon-exon junction, and the target probes in the pair of target probes may be on opposite sides of the exon junction.

The signal amplification molecule may be hybridized to a plurality of branch probes; wherein each branch probe is hybridized to a plurality of HRP probes; and each HRP probe may be conjugated to an HRP molecule.

In embodiments, in step i), the detectable fluorophore of each CFT is different.

The detectable fluorophores of the CFT may be selected from Cy3, Cy5, Cy7, CBT, and fluorescein.

The hapten of the CHT may be selected from biotin, fluorescein, TAMRA, DNP, and DIG.

The tissue may be a formalin-fixed paraffin-embedded tissue.

In another aspect, provided herein is a cleavable hapten tyramide (CHT) comprising formula I

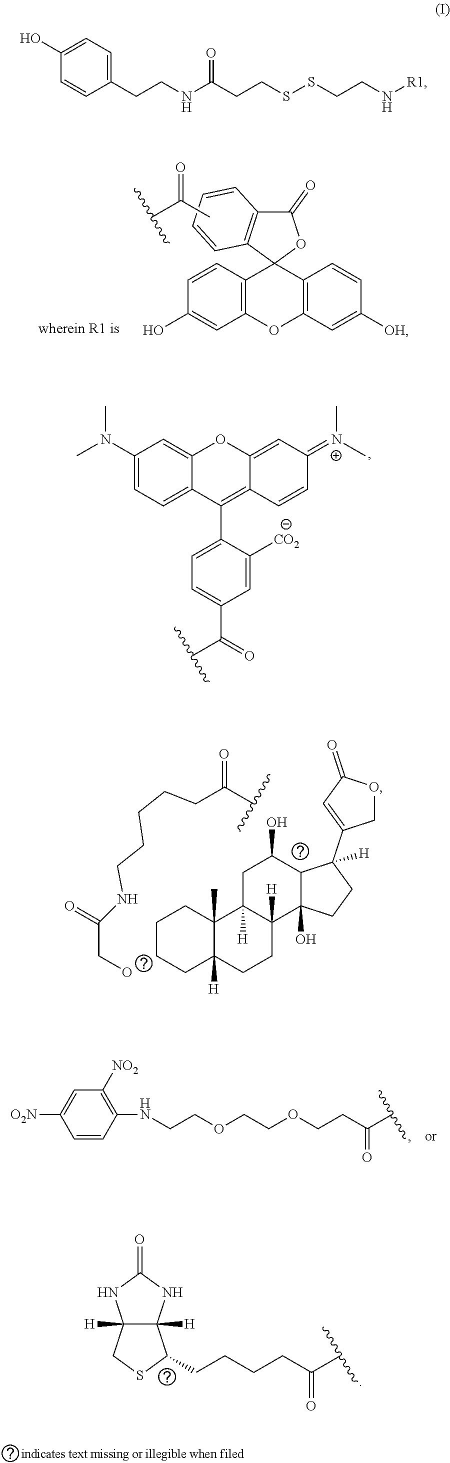

The CHT may comprise formula II

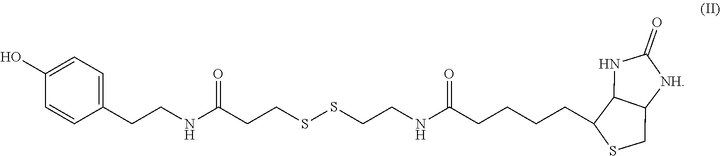 [SMILES: O=C(CCCCC1SCC2NC(=O)NC21)NCCSSCCC(=O)NCCc1ccc(O)cc1]

In embodiments, before step a), the tissue is contacted with a quencher. The quencher may be alkyne tyramide.

Before step f) the method may further comprise performing a click chemistry reaction.

### BRIEF DESCRIPTION OF THE DRAWINGS

The patent or patent application file contains at least one drawing in color. Copies of this patent or patent application publication with color drawings will be provided by the Office upon request and payment of the necessary fee.

The present disclosure will be better understood and features, aspects, and advantages other than those set forth above will become apparent when consideration is given to the following detailed description thereof. Such detailed description makes reference to the following drawings.

FIGS. 1A-1D. Enhanced sensitivity and multiplexing in situ RNA detection using modified RNAScope® Multiplex Fluorescent v2 assay combined with cleavable biotin tyramide (CBT) and cleavable fluorescent tyramides (CFTs). (A) Schematic overview of modified RNAScope® workflow. Target mRNAs are first hybridized using probes from the RNAScope® Multiplex Fluorescent v2 kit. Signal amplification is achieved by incorporating CBT, followed by Horseradish Peroxidase (HRP)-conjugated streptavidin (SA-HRP) and CFTs. (B) Sequential detection of three mRNA targets. Each cycle targets three distinct mRNAs, visualized in separate fluorescent channels via three different CFTs. After developing the signal for the first target, HRP is inactivated, and the excess biotin-binding site on streptavidin is blocked with free biotin. Subsequent RNA targets are detected similarly. Once imaging for all targets is complete, chemical removal of biotin and fluorophores by cleavage solution, followed by stripping the DNA probes for further cycles of detection. (C) Imaging workflow for the modified RNAScope® assay. (D) Chemical structures of CBT (tyramide-S—S-biotin) and CFTs (Tyramide-N₃-fluorescein, Tyramide-N₃-Cy3, and Tyramide-N₃-Cy5).

FIGS. 2A-2E. Evaluation of 3-Plex Positive Control Probe Staining with and without CBT Amplification (A) Staining results using the standard RNAscope® Multiplex Fluorescent V2 assay. Human FFPE brain tissue was stained with 3-plex positive control probes targeting Polr2a, PPIB, and UBC. Detection was achieved with three distinct cleavable CFTs: tyramide-N₃-FITC, tyramide-N₃-Cy3, and tyramide-N₃-Cy5, respectively. (B) Staining results with CBT amplification. The same 3-plex control probes were applied with additional amplification using CBT in combination with the same CFTs, enhancing the fluorescent signal in human FFPE brain tissue. (C) Comparison of fluorescence signal intensity. Fluorescence intensity profiles from standard RNAscope® (green line) and CBT-amplified RNAscope® (red line) are shown, with white dots representing detected mRNA transcripts. DAPI-stained nuclei appear in blue. (D) Quantification of mean intensity per spot. The mean fluorescence intensity for each spot was measured in the three channels (FITC, PPIB, and Cy5), with and without CBT amplification (n=30 cells). (E) Comparison of transcript copy number per cell. Spot counts per cell in the three different channels (FITC, PPIB, and Cy5) were compared between the standard RNAscope® assay and the CBT-amplified assay. Scale bar: 50 μm.

FIGS. 3A-3C. Assessing crosstalk between fluorescent channels in sequential mRNA staining cycles. (A) Sequential staining of SLC32a1, S100B, and ACHE mRNAs using tyramide-N₃-FITC, tyramide-N₃-Cy3, and tyramide-N₃-Cy5, respectively, in a single staining cycle. Scale bar: 30 μm. (B) Sequential staining of six mRNAs in two reiterative cycles. In the first cycle, SLC32a1, S100B, and ACHE mRNAs were stained in the FITC, Cy3, and Cy5 channels, respectively (left panels, with white dots indicating staining). After image acquisition, the fluorophores were removed, and the DNA probes were stripped. In the second cycle, SLC17a7, GFAP, and Olig1 mRNAs were stained in the same FITC, Cy3, and Cy5 channels, respectively (middle panels, with white dots indicating staining). An overlay of the two cycles shows the first staining in green dots and the second staining in magenta dots. (right panels) DAPI-stained nuclei are shown in blue. (Scale bar: 30 μm) (C) Fluorescence intensity profiles of sequential staining cycles. The intensity profiles for the first and second staining cycles are shown in green and magenta, respectively, demonstrating the absence of significant crosstalk between the two rounds of staining.

FIGS. 4A-4B. Sequential detection and spatial mapping of ten target mRNAs in human FFPE brain tissue. (A) Sequential detection of ten mRNAs. Ten distinct target mRNAs were detected using the RNAscope® Multiplex Fluorescent v2 assay, enhanced by additional CBT amplification, and visualized with three different CFTs in human FFPE brain tissue. (Scale bar: 50 μm) (B) Spatial localization of mRNA counts per cell in the human cerebral cortex. The number of mRNA transcripts per cell for the ten different targets was quantified and mapped using the image analysis software CellProfiler, showing the distribution of transcript counts across individual cells in the cerebral cortex.

FIGS. 5A-5C. Identification and visualization of neural-specific cell clusters in the human cerebral cortex using k-NN and Leiden algorithms on UMAP plot. (A) UMAP plot of 26,040 cells. The cells were classified into 11 distinct clusters based on k-nearest neighbors (k-NN) graph and the Leiden algorithm. (B). Spatial localization of clusters in the human cerebral cortex. The 11 identified clusters were mapped back to their original locations within the human cerebral cortex. (Scale bar: 1000 μm) (C) Distinct gene expression patterns across cell clusters. The z-scores of gene expression reveal unique profiles in the 11 clusters: Cluster 01: Dopaminergic neurons (NR4a2 enriched), Cluster 02: GABAergic neurons (SLC32a1 enriched), Cluster 03: Glutamatergic neurons (SLC17a7 enriched), Cluster 04: excited cholinergic neurons (ACHE&SLC17a7 enriched), Cluster 05: inhibitory cholinergic neurons (ACHE&SLC32a1 enriched), Cluster 06: Microglia (AIF1 enriched), Cluster 07: Serotonergic neurons (TPH1 enriched), Cluster 08: Astrocytes (S100B enriched), Cluster 09: Astrocytes (GFAP enriched), Cluster 10: Oligodendrocytes (Olig1&S100B enriched), and Cluster 11: Oligodendrocytes (Olig1 enriched).

FIG. 6. Neighborhood analysis of 11 neural clusters. The color bar represents the odds ratio, indicating the likelihood of cells from other clusters being located within a 75 μm radius of a central cell from a given cluster. A positive odds ratio suggests that the neighboring cluster is more likely to appear in the proximity of the central cluster cell, while a negative odds ratio indicates a lower likelihood of co-localization.

FIGS. 7A-7C. Subcluster analysis of Cluster 09 (GFAP-enriched Astrocytes). (A) Identification of three subclusters. Three subclusters (9A in red, 9B in green, and 9C in blue) were identified semi-manually on a UMAP plot from 1,268 cells in Cluster 09, based on the composition of neighboring cells. (B) Spatial localization of subclusters in the human cerebral cortex. The positions of the three subclusters within Cluster 09 are mapped in the cerebral cortex tissue. (C) Average cell numbers in each subcluster. The average number of cells from each major cluster within the three subclusters is shown. The color bar represents the cell count of nearby clusters.

FIGS. 8A-8B. The positive and negative control of RNAscope® Multiplex Fluorescent v2 Assay with additional CBT amplification. (A) 3-plex positive control probes (C1-Polr2a, C2-PPIB, and C3-UBC) and (B) 3-plex negative control probes (C1-DapB, C2-DapB, and C3-DapB stained with three different CFTs (tyramide-N₃-FITC, tyramide-N₃-Cy3, and tyramide-N₃-Cy5) in human FFPE cerebral cortex tissue. Scale bar, 50 μm. The staining of mRNAs is shown in white dots. The nuclei stained with DAPI are shown in blue.

FIG. 9. Z-normalized RNA expression level of nine neural markers visualized on UMAP plots. UMAP plots displaying the Z-normalized expression levels of nine neural markers in human FFPE cerebral cortex tissue. Each plot represents the expression distribution of a specific neural marker across all cells, with color intensity indicating relative expression levels. The dimensionality reduction highlights distinct neural cell clusters based on marker expression profiles.

FIGS. 10A-10B. (A) Anatomical distribution of individual cells from 11 defined clusters in the human FFPE cerebral cortex tissue. Clusters are color-coded to indicate their spatial organization. (Scale bar: 1000 μm) (B) Representative RNA-FISH staining of the 11 annotated clusters, with green and magenta spots indicating the presence of corresponding mRNAs. Distinct staining patterns highlight the cellular heterogeneity within the tissue. (Scale bar: 20 μm).

FIG. 11. Synthetic Scheme of Tyramide-S—S-Biotin.

FIGS. 12A-12D. (A) RNA isoform staining by proximity labeling probes. (B) After click chemistry and cleavage, only specific exon staining signals remain. (C) Chemical structure of cleavable biotin tyramide. (D) Click chemistry reaction to quench the cleavable linker.

FIG. 13. Optimization of the paired target recognition probes. RNA, target recognition probes and amplification probes are shown in purple, green and red, respectively.

FIG. 14. Exon junction staining with cleavable biotin tyramide and cleavable fluorescent tyramide.

FIG. 15. Exon junction staining with amplification probes 1a and 1b continuously hybridized to each other.

FIG. 16. Ultrasensitive RNA staining with 1 pair of target probes using click-chemistry and background removal. Human FFPE melanoma tissue was first stained by RNAscope (gold standard) with 20 pairs of target hybridization probes (first line). In the second cycle, only 1 pair of target hybridization probes was applied to detect POLR2A, PPIB, and UBC (second line). In the images, red signals represent RNAscope staining from the first cycle, while green signals correspond to the staining signals from the second cycle. SATA FISH stands for signal amplification triggered amplification (concept illustrated in FIG. 14). Following click reaction to couple the alkyne quencher deposited in cycle 1 and cleavable fluorophores deposited in cycle 2, background cleavage is carried out (third line). Overlay images demonstrate colocalization of signals generated by our approach and RNAscope (fourth line). Arrows a-f indicate representative regions used for intensity analysis. Regions overlapping with RNAscope signals exhibit minimal fluorescence loss after cleavage whereas background regions display substantial signal reduction.

FIG. 17. Negative control of SATA-FISH using only 1 hybridization probe. Human FFPE melanoma tissue was first stained by RNAscope (red) using cleavable fluorescent tyramide and alkyne tyramide as a reference (first line). SATA-FISH was then performed using only 1 target probe from the original probe pair (second line). Overlay images show no specific colocalization with RNAscope signals (third line). Following click reaction to couple the alkyne quencher deposited in cycle 1 and cleavable fluorophores deposited in cycle 2, background cleavage is carried out (fourth line). Arrows a-f indicate representative regions used for intensity analysis. Following click reaction and fluorophore cleavage, complete signal removal was observed in both RNAscope signal regions and background regions, indicating uniformly high cleavage efficiency under single-probe conditions.

FIG. 18. Ultrasensitive and multiplexed RNA isoform imaging. The RNA target is first labeled with a quencher and then restained for a specific exon using cleavable fluorophores. A proximity coupling reaction deactivates the cleavable linker close to the quencher. The subsequent chemical cleavage only removes the nonspecific staining signals. After imaging and signal removal, the next cycle is initiated.

FIG. 19. Synthesis of cleavable fluorescent tyramide with varied linker length.

FIGS. 20A-20B. (A) Chemical structures of cleavable hapten tyramide. (B) Chemical structures of cleavable fluorescent tyramide.

### DETAILED DESCRIPTION

Accurate detection and localization of RNA within cells is critical for understanding gene expression, regulation, and RNA-protein interactions in various biological contexts. RNA fluorescent in situ hybridization (RNA-FISH) has emerged as a powerful tool for visualizing single RNA molecules in complex tissue with high spatial resolution, offering insights into the subcellular distribution and dynamics of specific RNA species. However, current RNA-FISH approach lacks proper signal amplification approaches for detecting low-abundance mRNAs in highly autofluorescent formalin-fixed paraffin-embedded (FFPE) tissues. To overcome this limitation, the inventors developed an ultrasensitive, multiplexed RNA profiling assay using a multiple-round tyramide signal amplification (TSA) approach, facilitating the detection of low-expressed mRNAs with green and orange fluorophores in FFPE tissues. Using this method, they successfully detected ten distinct mRNAs in human FFPE cerebral cortex tissue, including low-abundance neural markers. In addition, approximately 26,000 cells were classified into specific neural cell clusters based on collected neural mRNA expression patterns. Furthermore, spatial analysis of neighboring cell compositions allowed for identification of unique subclusters within each neural cell type, offering deeper insights into their microenvironments. Thus, this highly sensitive and multiplexed RNA profiling platform provides a powerful tool for comprehensive RNA profiling, advancing our understanding of complex biological systems. Additionally, the inventors developed a highly multiplexed short RNA imaging approach, which enables comprehensive single-cell short RNA analysis in intact cells and tissues in situ.

In a first aspect, provided herein is a method for multiplexed in situ analysis of nucleic acids in a tissue, the method comprising: a) contacting the tissue with a target probe or a pair of target probes capable of hybridizing to a target nucleic acid; b) contacting the tissue with a signal amplification molecule capable of binding to the target probe or pair of target probes, wherein the signal amplification molecule comprises a plurality of horseradish peroxidase (HRP) molecules; c) contacting the tissue with a cleavable hapten tyramide (CHT) in a tyramide signal amplification (TSA) buffer; d) contacting the tissue with HRP-conjugated streptavidin (SA-HRP); e) contacting the tissue with a cleavable detectably-labeled tyramide in a TSA buffer; f) imaging the tissue; g) contacting the tissue with an HRP deactivating agent; h) contacting the tissue with biotin; i) repeating steps a) through h) for a plurality of rounds, wherein in each round, the target probe is capable of hybridizing to a different target nucleic acid; and wherein the fluorophore of each CFT is different than an immediately previous round.

The tissue may be a formalin-fixed paraffin-embedded tissue. Any fixative (e.g. formaldehyde, or paraformaldehyde) that does not affect antibody binding or nucleic acid hybridization can be utilized. In other embodiments, the methods are performed on unfixed (“fresh”) tissue samples.

The tissue may be a sample from a biological entity. The term “biological entity” as used herein means any independent organism or thing, alive or dead, containing genetic material (e.g., nucleic acid) that is capable of replicating either alone or with the assistance of another organism or cell. Sources for nucleic acid-containing biological entities include, without limitation, an organism or organisms including a cell or cells, bacteria, yeast, fungi, algae, viruses, or a sample thereof. Specifically, an organism of the current disclosure includes bacteria, algae, viruses, fungi, and mammals (e.g., humans, non-human mammals). The methods and compositions described herein can be performed using a variety of biological or clinical samples comprising cells that are in any (or all) stage(s) of the cell cycle (e.g., mitosis, meiosis, interphase, G0, G1, S and/or G2). As used herein, the term “sample” include all types of cell culture, animal or plant tissue, peripheral blood lymphocytes, buccal smears, touch preparations prepared from uncultured primary tumors, cancer cells, bone marrow, cells obtained from biopsy or cells in bodily fluids (e.g., blood, urine, sputum and the like), cells from amniotic fluid, cells from maternal blood (e.g., fetal cells), cells from testis and ovary, and the like. In some cases, samples are obtained by swabbing, washing, or otherwise collecting biological material from a non-biological object such as a medical device, medical instrument, handrail, doorknob, etc. Samples are prepared for assays of this disclosure using conventional techniques, which typically depend on the source from which a sample or specimen is taken. These examples are not to be construed as limiting the sample types applicable to the methods and/or compositions described herein.

As used herein, the terms “nucleic acid” or “oligonucleotide” refer to and encompass any physical string or collection of monomer units (e.g., nucleotides) that can connect to form a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the nucleic acid can be deoxyribonucleotides, ribonucleotides or nucleotide analogs, and can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The nucleic acid can additionally comprise non-nucleotide elements such as labels, quenchers, blocking groups, or the like. The nucleic acid can be single-stranded or double-stranded.

As used herein, the terms “nucleic acid of interest,” and “target nucleic acid” include a nucleic acid originating from one or more biological entities within a sample. The target nucleic acid of interest to be detected in a sample can be a sequence or a subsequence from DNA, such as nuclear or mitochondrial DNA, or cDNA that is reverse transcribed from RNA in the sample. The sequence of interest can also be from RNA, such as mRNA, IRNA, tRNA, miRNA, siRNAs, antisense RNAs, or long noncoding RNAs. More generally, the sequences of interest can be selected from any combination of sequences or subsequences in the genome or transcriptome of a species or an environment. In some cases, a defined set of targeting agents are oligonucleotide probes that are designed to hybridize to the plurality of sequences that would be expected in a sample, for example a genome or transcriptome, or a smaller set when the sequences are known and well-characterized, such as from an artificial source. In embodiments, the target nucleic acid comprises an exon-exon junction. An exon-exon junction is the site where two exons are joined together after introns are removed during pre-mRNA splicing. In embodiments, the two target probes of a pair are capable of hybridizing to the target nucleic acid at opposite sides of the exon-exon junction.

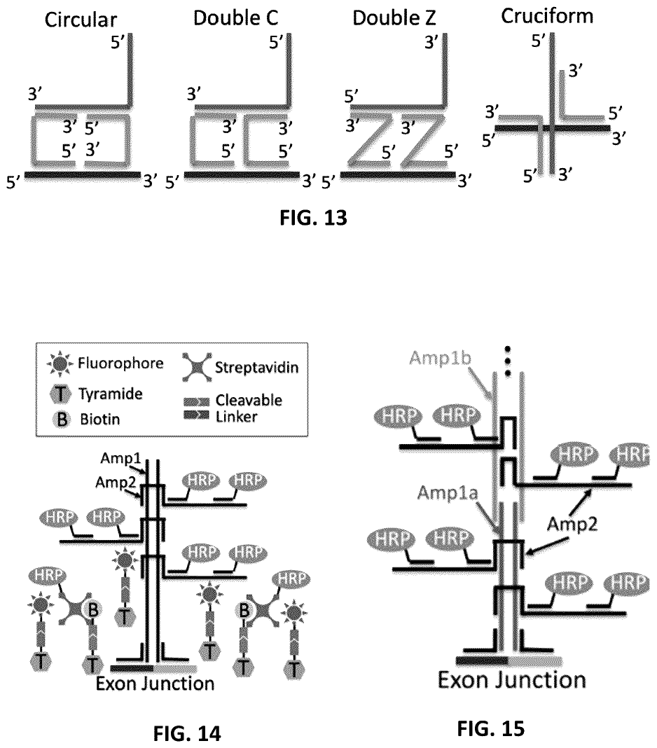

The term “target probe” refers to a nucleic acid molecule that is capable of hybridizing to a target nucleic acid. The method may employ a pair of target probes. The pair of target probes may comprise “Z” probes, in which each probe comprises a target-specific sequence that binds to the target nucleic acid, a spacer, and a tail sequence. When the double Z probes bind the target nucleic acid, the tail sequences for a hybridization site for the signal amplification molecule. The Z type target probes and their use in RNAscope are described in U.S. Pat. No. 7,709,198 B2, which is incorporated herein by reference. Other target probe pair designs and orientations, e.g. circular, double C, and cruciform, are illustrated at FIG. 13.

The terms “hybridize” and “hybridization” as used herein refer to the association of two nucleic acids to form a stable duplex. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, N.Y.). One of skill in the art will understand that “hybridization” as used herein does not require a precise base-for-base complementarity. That is, a duplex can form, between two nucleic acids that contained mismatched base pairs. The conditions under which nucleic acids that are perfectly complementary or that contain mismatched base pairs will hybridize to form a duplex are well known in the art and are described, for example, in MOLECULAR CLONING: A LABORATORY MANUAL, 3^{rd} ed., Sambrook et al., eds., Cold Spring Harbor Press, Cold Spring Harbor (2001) at Chapter 10, which is herein incorporated by reference. As used herein, the term “complementary” refers to a nucleic acid that forms a stable duplex with its “complement”. For example, nucleotide sequences that are complementary to each other have mismatches at less than 20% of the bases, at less than about 10% of the bases, preferably at less than about 5% of the bases, and more preferably have no mismatches.

The term “signal amplification molecule” is a nucleic acid molecule capable of hybridizing to at least one target probe. The signal amplification molecule comprises a plurality of HRP molecules. In embodiments, the signal amplification molecule is hybridized to a plurality of branch probes, wherein each branch probe is further hybridized to a plurality of HRP probes, wherein each HRP probe is conjugated to an HRP molecule. The plurality of branch probes result in a branched effect, which provides physical space for the loading of multiple HRP probes and HRP molecules onto the signal amplification molecule.

In embodiments, the signal amplification molecule is hybridized to at least one additional signal amplification molecule, each hybridized to a plurality of branch probes and HRP probes.

A cleavable hapten tyramide (CHT) is a molecule comprising a hapten and a tyramide joined by a cleavable linker. In exemplary embodiments, the cleavable linker is a disulfide bond. Suitable haptens for use in the CHT include, without limitation, biotin, fluorescein, Tetramethylrhodamine (TAMRA), dinitrophenol (DNP), and digoxigenin (DIG). The CHT may comprises formula I

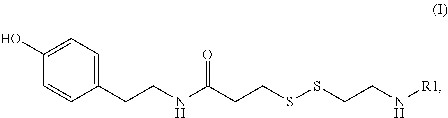

where R1 is

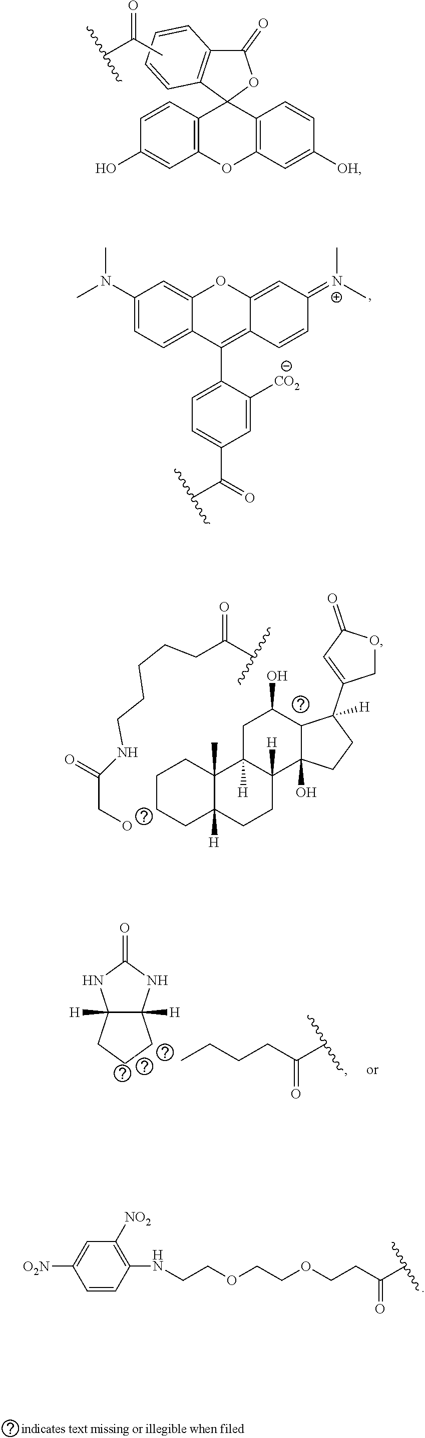

In exemplary embodiments, the CHT is a cleavable biotin tyramide (CBT) having formula II

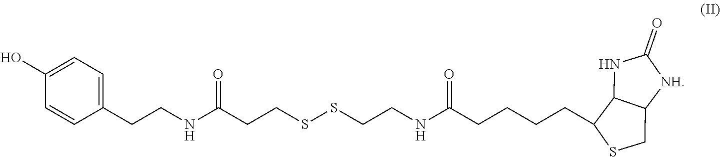 [SMILES: O=C(CCCCC1SCC2NC(=O)NC21)NCCSSCCC(=O)NCCc1ccc(O)cc1]

A tyramide signal amplification (TSA) buffer is a buffer that utilizes the catalytic activity of horseradish peroxidase (HRP) for high-density labeling of a target nucleic acid in situ.

In certain embodiments, a detectable label such as a fluorophore is tethered to tyramide via a cleavable linker. Preferably, the cleavable linker is a chemically cleavable linker. As described herein, to enable signal removal (e.g., fluorescent signal) after protein staining the cleavable detectably labeled tyramide preferably comprises a fluorophore tethered to tyramide through a chemically cleavable linker. An important aspect of the technology of this disclosure is efficient cleavage of the detectable label in a cellular environment while maintaining protein antigenicity. Additionally, it is preferred that the linker is small enough to permit recognition of CLT by horseradish peroxidase (HRP) and to avoid compromised diffusion of a short-lived tyramide radical. In embodiments, the cleavable linker comprises the structure of Formula III, wherein R comprises a detectable marker and T comprises tyramide:

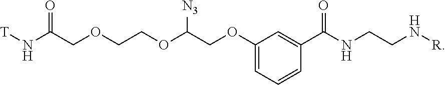

A cleavable fluorophore tyramide (CFT) is a molecule comprising a fluorophore and a tyramide joined by a cleavable linker. Suitable fluorophores for use in the CFT include, without limitation, Cy2, Cy 3, Cy3.5, Cy 5, Cy5.5, Cy7, TAMRA (labeled with tetramethylrhodamine or “TMR”), ALEXA FLUOR™ 594, and ATTO 647N and ATTO 700 fluorophores (ATTO-TEC, Germany). Other fluorophores appropriate for use according to the methods provided herein include, without limitation, quantum dots, ALEXA FLUOR™ 350, ALEXA FLUOR™ 532, ALEXA FLUOR 546, ALEXA FLUOR™ 568, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, DYLIGHT™ DYES (e.g., DYLIGHT™ 405, DYLIGHT™ 488, DYLIGHT™ 549, DYLIGHT™ 594, DYLIGHT™ 633, DYLIGHT™ 649, DYLIGHT™ 680, DYLIGHT™ 750, DYLIGHT™ 800 and the like), and Texas Red. In addition to the use of fluorophores as a detectable moiety, other labels such as luminescent agents (e.g., chemiluminescent agents), fluorescent proteins, and radioisotopes can also be used as detection tags.

Other cleavable linkers appropriate for use in a CFT of this disclosure include, structures cleaved by enzymes, nucleophiles, electrophiles, reducing reagents, oxidizing reagents, photo-irradiation, metal catalysis, and the like. Further examples of suitable linkers and cleavage mechanisms are described by Milton et al. (U.S. Pat. No. 7,414,116) and by Leriche et al. (Bioorg. Med. Chem., 2012, 20:571-582), which are incorporated herein by reference in their entirety. The linker may be cleavable using a variety of approaches including the addition of a chemical agent, irradiation with one or more wavelengths of light, enzymatic reaction and the like.

Cleavable detectably labeled tyramides are disclosed in U.S. Publication No. 2023-0055832 A1, which is incorporated by reference.

Fluorescence photomicroscopy can be used to detect and record the results of consecutive in situ analysis using routine methods known in the art. Alternatively, digital (computer implemented) fluorescence microscopy with image-processing capability may be used.

An HRP deactivating agent is a reagent that inactivates HRP enzymes after tyramide signal amplification.

Step a) (contacting the tissue with target probes) may be done for between about 0.1 and about 48 hours and at between about 15 and about 60° C. In exemplary embodiments, step a) is done for about 2 hours at about 40° C.

Step b) (contacting the tissue with a signal amplification molecule) may be done for between about 1 and about 300 minutes and at between about 15 and about 60° C. In exemplary embodiments, step b) is done for about 30 minutes at about 40° C.

Step c) (contacting the tissue with a cleavable hapten tyramide (CHT) in a TSA buffer) may be done for between about 1 and about 300 minutes and at between about 15 and about 60° C. In exemplary embodiments, step b) is done for about 30 minutes at about 40° C.

Between steps c) and d), the method may further comprise washing the tissue in a buffer. The tissue may be washed for about 2 minutes at about room temperature (20° C.-23° C.). The tissue may be washed about two times.

Step d) contacting the tissue with SA-HRP) may be done for between about 1 and about 300 minutes and at between about 15 and about 60° C. In exemplary embodiments, step b) is done for about 30 minutes at about room temperature.

Step e) (contacting the tissue with a cleavable fluorophore tyramide (CFT) in a TSA buffer) may be done for between about 1 and about 300 minutes and at between about 15 and about 60° C. In exemplary embodiments, step e) is done for about 30 minutes at about 40° C.

Steps d) and e) may be done in an incubation buffer comprising 2×SSC, 1 mg/mL BSA, and 0.1% Tween-20.

The tissue may be washed for about 2 minutes between steps e) and f). The tissue may be washed about two times.

Step g) (contacting the tissue with an HRP deactivating agent) may be done for between about 1 and about 300 minutes and at between about 15 and about 100° C. In exemplary embodiments, step g) is done for about 15 minutes at about 40° C.

Step h) (contacting the tissue with biotin) may be done for between about 1 and about 300 minutes and at between about 15 and about 60° C. In exemplary embodiments, step h) is done for about 30 minutes at about room temperature.

In the method described herein the steps a) through h) may be repeated for a plurality of rounds. For example, the steps may be repeated once, twice, three times, four times, five times, etc. In each round, the fluorophore used in the CFT may be different than the immediately previous round, i.e. the round right before the current round. The CFT may be different in every round.

The method may further comprise j) contacting the tissue with a first cleavage solution comprising tris(2-carboxyethyl)phosphine (TCEP); k) contacting the tissue with a second cleavage solution comprising 1,3,5-Triaza-7-phosphaadamantane (PTA); 1) contacting the tissue with DNase I; m) contacting the tissue with a DNase I quenching buffer; n) contacting the tissue with formamide; o) repeating steps a) through n). In embodiments, steps j) and k) are performed together. A DNase I quenching buffer comprises 30% formamide and 0.1% Tween-20 in 2×SSC.

Step j) (contacting the tissue with TCEP) may be done for between about 1 and about 300 minutes and at between about 15 and about 100° C. In exemplary embodiments, step j) is done for about 30 minutes at about 40° C.

Step k) (contacting the tissue with PTA) may be done for between about 1 and about 300 minutes and at between about 15 and about 100° C. In exemplary embodiments, step k) is done for about 30 minutes at about 40° C.

Step l) (contacting the tissue with DNase I) may be done for between about 1 and about 300 minutes and at between about 15 and about 60° C. In exemplary embodiments, step 1) is done for about 1 hour at about room temperature.

Step m) (contacting the tissue with DNase I quenching buffer) may be done for between about 1 and about 300 minutes and at between about 15 and about 60° C. In exemplary embodiments, step m) is done for about 20 minutes. Step m) may be done about two times.

Step n) (contacting the tissue with 70% formamide) may be done for between about 1 and about 300 minutes and at between about 15 and about 100° C. In exemplary embodiments, step n) is done for about 30 minutes at about 40° C.

In alternative embodiments, steps l)-n) are replaced with any suitable method for stripping the probes from the tissue.

The term “stripping” encompasses using heat, a buffer, denaturing agent, detergent, temperature condition, pH condition, osmolarity agent, or any combination thereof. Non-limiting examples of stripping include boiling a sample or subjecting a sample to a boiling solution, subjecting a sample to a very low pH (e.g. pH 2) or high pH (e.g. pH 10), and/or subjecting a sample to a denaturing agent or detergent, such as, e.g., subjecting a sample to SDS, a glycine buffer, glycine SDS, and/or boiling a sample in citrate buffer. Heat-induced stripping may optionally be accomplished by microwaving a sample or subjecting a sample to solution which has recently been microwaved or otherwise brought to its boiling point. For example, heat-induced stripping encompasses subjecting a sample to a 100° C. condition for at least about 1 minute, 5 minutes, 10 minutes, 20 minutes, or greater. Stripping may optionally be accomplished using a combination of heat-induced stripping and another agent or condition, such as, e.g., a buffer, pH condition, osmolarity agent, detergent, denaturing agent, or any combination thereof.

The method may further comprise washing the tissue between steps n) and o).

In embodiments, nonspecific signal is reduced by a proximity coupling reaction. In such embodiments, before step a), the tissue is contacted with a quencher. The quencher may be alkyne tyramide

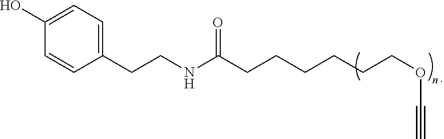

The method may further comprise performing a click chemistry reaction before step f). The click chemistry reaction may be done by contacting the tissue with about 0.6 mM CuSO4, about 3 mM THPTA, optionally in PBS, and about 4 mM of sodium ascorbate, optionally freshly made at about room temperature for about 30 minutes. The alkyne/quencher will click with the azide group in the cleavable fluorescent tyramide to make its fluorophore noncleavable. The cleavable linker on the specific signal is deactivated, and becomes uncleavable. But the nonspecific signals are still cleavable (and removed) as their cleavable linker (N3 group) is not reacted/quenched by the alkyne/quencher.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

### Example 1

### Introduction

In our previous study [24], we developed a method combining the RNAscope® Multiplex fluorescent v2 Assay [25] and cleavable fluorescent tyramide (CFT), enabling multi-cycle staining in both fresh frozen tissue and FFPE tissue. This platform has successfully detected eight different mRNAs in fixed frozen mouse brain tissue with cleavable far-red dye (tyramide-N₃-Cy5, 650 nm). Building on the findings of previous research, the present study aims to expand the range of usable fluorophores and address high autofluorescence in the green (430 nm) and orange (550 nm) channels, which are major obstacles to effective fluorescence detection in FFPE tissue. To achieve this, our approach incorporates an additional tyramide signal amplification (TSA) step using cleavable biotin tyramide (CBT), which recruits more horseradish peroxidase (HRP)-conjugated streptavidin (SA-HRP). The increased deposited HRP leads to a greater accumulation of fluorophores, thereby enhancing signal intensity and detection sensitivity. In this multiple-round TSA approach, target mRNAs are first recognized by double Z target probes and amplified by amplification probes from RNAscope® Multiplex Fluorescent v2 Assay. After channel-specific HRP-conjugated oligonucleotide hybridizes to amplification probes, the first TSA reaction deposits of CBT, recruiting additional SA-HRP. A second TSA reaction deposits the corresponding CFT to complete the first target signal development. Before developing the subsequent channel signal, the deposited SA-HRP and the oligonucleotide HRP are deactivated using an HRP blocker from the RNAscope® kit. The streptavidin in SA-HRP is then blocked by free biotin. After capturing fluorescence images from all three channels, the deposited biotins and fluorophores are chemically cleaved, and the DNA probes are stripped using DNase I and a high concentration of formamide, enabling the next cycle staining.

To validate this platform, ten mRNAs with diverse expression levels were quantified in situ through reiterative cycles of target probe hybridization, fluorescence signal development, fluorescence imaging, fluorophore cleavage, and probe removal in human FFPE cerebral cortex tissue. Based on the derived nine neural mRNA expression profiles, we clustered 26,040 cells into eleven distinct clusters, each corresponding to specific neural phenotypes. Additionally, we observed unique cellular neighborhood compositions in specific neural clusters, providing valuable insights into the intricate cellular architecture of human brain tissue. This method provides a robust and versatile platform for in situ gene expression studies, offering extraordinary signal amplification to overcome the high autofluorescence background in FFPE tissue. By enabling the use of three-color fluorophores, it allows for precise detection of mRNAs with diverse expression levels, facilitating more comprehensive and reliable molecular analysis. This technique holds broad potential for clinical diagnostics and basic research, particularly in unraveling the molecular mechanisms underlying complex diseases.

### Materials and Methods

### General Information

Chemicals and solvents were obtained from TCI America (Portland, OR, USA) or Sigma-Aldrich (St. Louis, MO, USA) and used without further purification. Bioreagents were purchased from G-biosciences (St. Louis, MO, USA), Invitrogen (Waltham, MA, USA), Thermo Fisher Scientific™ (Waltham, MA, USA), Roche (Basel, Switzerland), and Advanced Cell Diagnostics (Newark, CA, USA), unless otherwise noted. Human cerebral cortex FFPE tissue (Cat #T2234042) was acquired from BioChain (Newark, CA, USA).

### Human Cerebral Cortex FFPE Tissue Pretreatment

Human cerebral cortex FFPE tissue slides were baked at 60° C. for 1 h. The slide was deparaffinized with two treatments in fresh xylene, 5 min each, followed by two washes with 100% ethanol, 2 minutes each. The slide was then dried at 60° C. for 5 min. To quench endogenous peroxidases, the tissue was incubated with RNAscope® hydrogen peroxidase (Advanced Cell Diagnostics, Newark, CA, USA) at room temperature for 10 min, then rinsed twice with distilled water. Following this, the slide was treated with RNAscope® target retrieval reagent (1×) (Advanced Cell Diagnostics, Newark, CA, USA) in a steamer at 100° C. for 30 min. After dehydration in 100% ethanol for 3 min, the slide was dried at 60° C. for 5 min. A hydrophobic barrier was created around the tissue sections using the ImmEdge® hydrophobic barrier pen (Vector Laboratories, Scottsdale, AZ, USA). The tissue was then digested with RNAscope® Protease Plus (Advanced Cell Diagnostics, Newark, CA, USA) at 40° C. for 30 min, followed by two washes with distilled water. After digestion, the tissue was immediately processed for target probe hybridization.

Multiplexed RNA Profiling with CBT Amplification and CFTs

After pretreatment, the human cerebral cortex FFPE tissue was incubated with the RNAscope® mRNA target probes (Advanced Cell Diagnostics, Newark, CA, USA) for 2 h at 40° C. The slide was processed using the RNAscope® Multiplex Fluorescent Reagent Kit v2 (Advanced Cell Diagnostics, Newark, CA, USA) according to the manufacturer's instructions. After the first channel HRP applied, signal amplification was conducted by incubating the slide with 2.5 μM cleavable biotin tyramide (tyramide-S—S-Biotin) in TSA amplification buffer (Advanced Cell Diagnostics, Newark, CA, USA) at 40° C. for 30 min. The slide was washed twice with 1× RNAscope® Wash Buffer (Advanced Cell Diagnostics, Newark, CA, USA) for 2 min at room temperature, followed by incubation with 5 μg/mL HRP-conjugated streptavidin (SA-HRP, Invitrogen, Waltham, MA, USA) in incubation buffer (2×SSC, 1 mg/mL BSA, and 0.1% Tween-20) for 30 min at room temperature. The fluorescent signal was developed by incubating the slide with 0.5 μm cleavable fluorophore tyramides (CFTs) in TSA amplification buffer (Advanced Cell Diagnostics, Newark, CA, USA) at 40° C. for 30 min, followed by two washes with 1× RNAscope® Wash Buffer for 2 min each. After capturing the fluorescent images, the slide was incubated with RNAscope® HRP Blocker (Advanced Cell Diagnostics, Newark, CA, USA) at 40° C. for 15 min for deactivation of deposited HRP. This was followed by incubation with 0.5 mg/mL of free biotin in 2×SSC at room temperature for 30 min. Subsequently, the channel HRPs for the other mRNA targets were applied to the tissue sequentially, and the same signal development procedure was repeated to complete the signal development of all three channels. The corresponding CFTs for C1, C2, and C3 channel HRP are Tyramide-N3-fluorescein, Tyramide-N3-Cy3, and Tyramide-N3-Cy5, respectively. The synthesis of these three CFTs is described in a previous publication.

### Fluorophore Cleavage and HRP Deactivation

After imaging all three channels, the fluorescent signal was removed between detection cycles by incubating the slide with cleavage solution 1 (100 mM TCEP in 5×SSC, pH 7.0) at 40° C. for 30 min, followed by incubation with cleavage solution 2 (100 mM PTA in 5×SSC, pH 8.0) at 40° C. for an additional 30 min. The oligonucleotide probes were stripped by incubating the slide with 0.5 unit/μL DNase I (Roche Diagnostics, Basel, Switzerland) at room temperature for 1 h, followed by quenching with DNase I quenching buffer (30% formamide and 0.1% Tween-20 in 2×SSC) twice for 20 min each. Any remaining probes were removed by incubating the slide in 70% formamide in 2×SSC at 40° C. for 30 min, followed by three washes in 1×PBS at room temperature. The slides were then prepared for the next cycle of RNA detection. The staining order and details of the probes used are provided in Table 1.

Table 1. The staining information of 10 different RNAscope® target probes. These probes are used for multiplexed single-cell in situ RNA staining in human cerebral cortex FFPE tissue.

| Staining order | Cycle | Channel | Probe name | Target cell type |
|---|---|---|---|---|
| 1 | 1 | FITC | Hs-NR4a2 | Dopaminergic neurons |
| 2 | 1 | Cy3 | Hs-TPH1 | Serotonergic neurons |
| 3 | 1 | Cy5 | Hs-AIF1 | Microglia |
| 4 | 2 | FITC | Hs-SLC32a1 | Glutamatergic neurons |
| 5 | 2 | Cy3 | Hs-S100B | Astrocytes |
| 6 | 2 | Cy5 | Hs-ACHE | Cholinergic neurons |
| 7 | 3 | FITC | Hs-SLC17a7 | GABAergic neurons |
| 8 | 3 | Cy3 | Hs-GFAP | Astrocytes |
| 9 | 3 | Cy5 | Hs-Olig1 | Oligodendrocytes |
| 10 | 4 | Cy5 | Hs-UBC | Housekeeping gene |

### Synthesis of the Cleavable Biotin Tyramide (CBT) and Cleavable Fluorescent Tyramides (CFTs)

Synthesis of Tyramide-S—S-Biotin: EZ-Link™ Sulfo-NHS—SS-Biotin (1 mg, 1.648 μmol; Thermo Fisher Scientific™ (Waltham, MA, USA)) was dissolved in 100 μL of anhydrous dimethylformamide (DMF) (Sigma-Aldrich (St. Louis, MO, USA)). In a separate vial, Tyramine-HCl (0.26 mg, 1.498 μmol) and N,N-Diisopropylethylamine (DIPEA) (0.291 mg, 2.248 μmol) (TCI America (Portland, OR, USA) were dissolved in 1 mL anhydrous of DMF and mixed thoroughly. A 26 μL aliquot of the Tyramine-HCl/DIPEA solution (containing 0.26 mg, 1.498 μmol of Tyramine-HCl and 0.291 mg, 2.248 μmol of DIPEA) was added to the Sulfo-NHS—SS-Biotin solution, and the mixture was stirred at room temperature for 2 h. The reaction was then quenched by adding 124 μL of ethanol and allowed to sit at room temperature for 15 min. The mixed product was directly used in staining applications without further purification.

### Imaging and Data Analysis

The stained FFPE tissue sections were imaged using a Nikon Ti-E 2 epifluorescent microscope equipped with a 20× objective (NA: 0.72). Fluorescence images were captured using an IRIS 15 camera (Teledyne Photometrics) with LED-DAPI-B, GFP-4050B, LED-TRIC-A, and LED-Cy5-5070A filters (Semrock). Image analysis and data processing were performed using NIS-Elements Imaging software (Nikon). Intensity quantification, pseudo-color processing, and image registration were accomplished using Fiji [26]. Cell segmentation and spot counting were carried out with CellProfiler [27] and QuPath [28]. Cell filtering, dimensionality reduction, and cell clustering were conducted using the OMIQ software (Dotmatics, www.omiq.ai) and SCANPY [29].

### Results and Discussion

### Platform Design

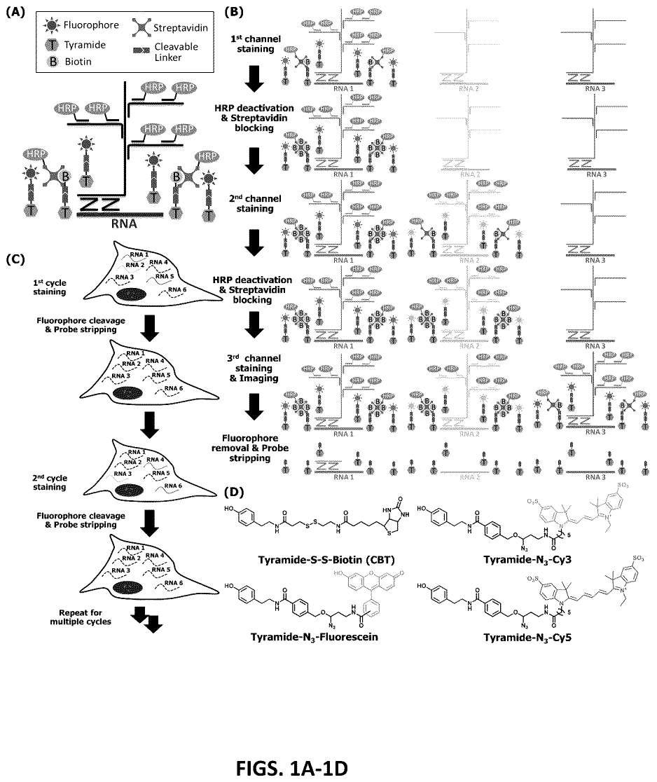

This multiplexed RNA profiling technique involves seven major steps in each staining cycle, as shown in FIG. 1. The target transcripts are initially hybridized with pairs of target-specific oligonucleotide probes (Step 1) (FIG. 1A.). Afterward, signal amplification via branched DNA assay (Step 2) occurs only when both independent probes in a pair successfully hybridize with the target probe in tandem. The processes of target hybridization and branched DNA amplification are performed simultaneously for all three RNA targets within each cycle. The first RNA target is further labeled by horseradish peroxidase (HRP) conjugated oligonucleotide. Each HRP enzyme is capable of catalyzing the deposition of up to 100 CBT molecules in close proximity to the target. (Step 3). Subsequently, the deposited biotin molecule attracts an SA-HRP, facilitating the deposition of up to 100 CFT molecules to develop a fluorescent signal for the first target (Step 4). After imaging, HRP molecules are deactivated using an HRP blocker, preserving RNA integrity, while deposited SA-HRP is blocked by free biotin (Step 5). Subsequently, the remaining two RNA targets are detected sequentially by repeating steps three through five with different colors of CFTs (Step 6) (FIG. 1B). At the end of each cycle, the deposited fluorophores and biotin are chemically removed using mild reducing agents, while the hybridized DNA probes are stripped using DNase I and a high-concentration formamide solution to prepare for the detection of the next set of RNA targets in the subsequent cycle (Step 7). By repeatedly performing cycles of target staining, fluorescence imaging, HRP deactivation, fluorophore removal, and probe stripping, multiple RNA targets can be profiled in the same tissue with single-cell resolution (FIG. 1C). The structures of the CBT and the three different colored CFTs used in this procedure are presented in FIG. 1D.

Enhancing RNAscope® v2 Kit Sensitivity with CBT Amplification

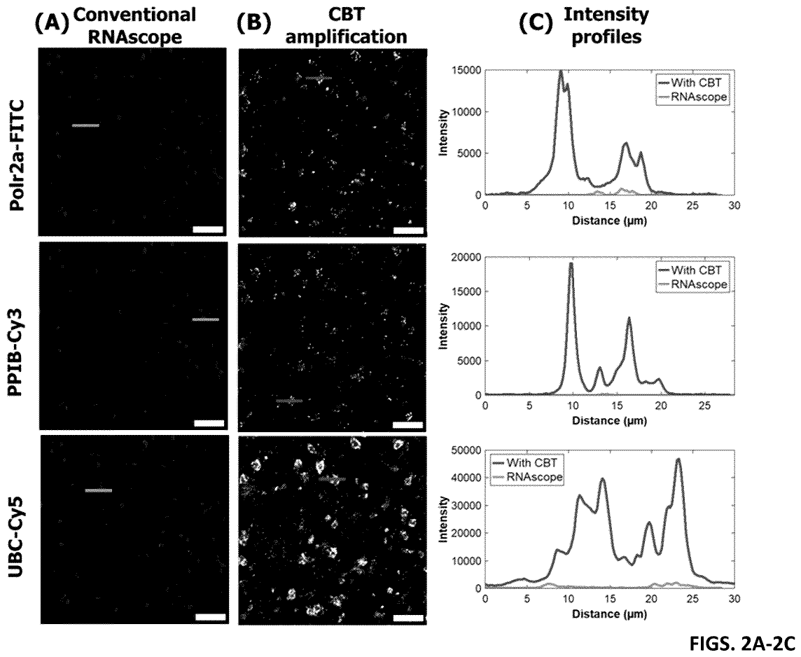

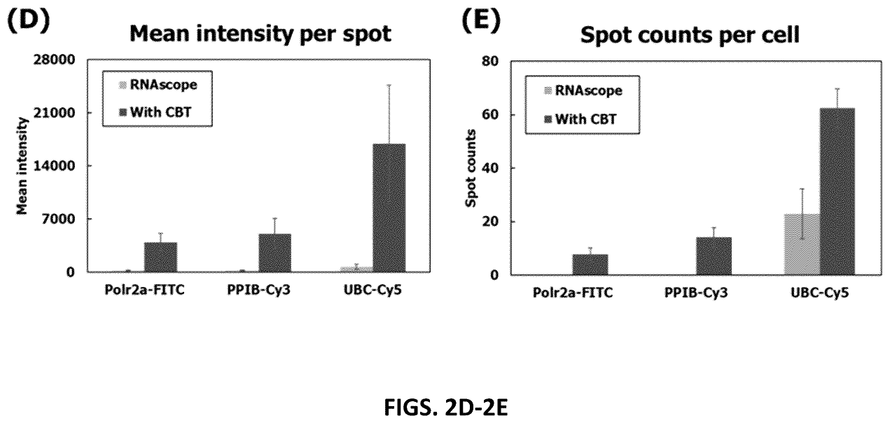

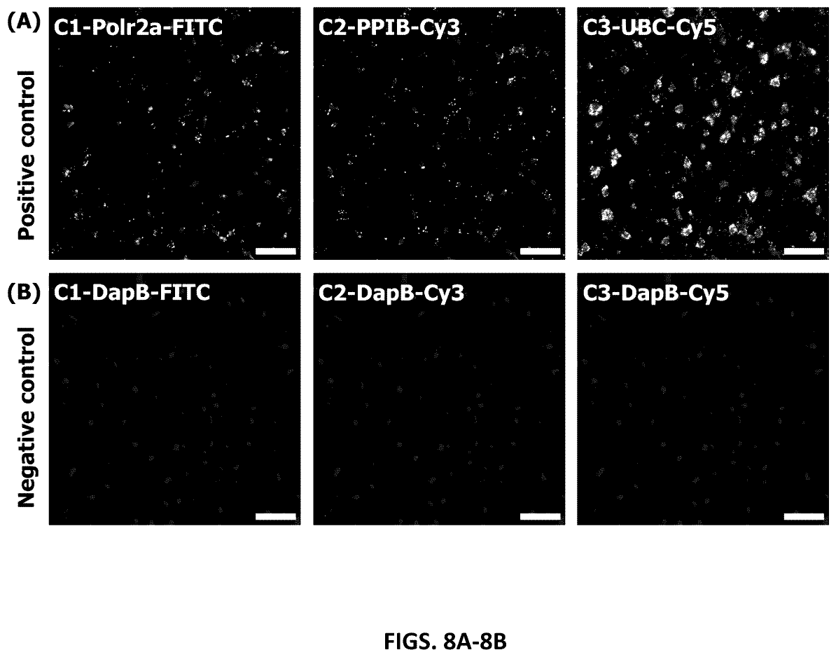

An enhancement of this method involves incorporating an additional TSA amplification step using CBT and SA-HRP (CBT amplification) to recruit more HRP, thereby significantly increasing the number of deposited fluorophores. To assess the level of signal enhancement achieved with the additional CBT amplification step, we compared the enhanced staining results to the conventional RNAscope® assay. This comparison utilized the 3-plex positive control probes (C1-Polr2a, C2-PPIB, and C3-UBC), stained with three different CFTs (tyramide-N₃-FITC, tyramide-N₃-Cy3, and tyramide-N₃-Cy5) in FFPE tissue from the human cerebral cortex. The compared results of the conventional RNAscope® assay and our additional CBT amplification approach are shown in FIG. 2. In the conventional RNAscope® assay results (FIG. 2A), the staining spots for the positive control probes were only visible in the Cy5 channel, displaying low staining intensity. In contrast to conventional, with the additional CBT amplification, distinct staining patterns for all three positive control probes were observed across all three channels (FIG. 2B), showing up to a 30-fold increase in signal intensity (FIGS. 2C and 2D). Furthermore, this signal amplification step not only enabled the detection of low-abundance mRNAs in the green and orange channels but also doubled spot counts of detectable mRNA in the Cy5 channel. (FIG. 2E). Along with this positive control staining, 3-plex negative control probes, a bacterial gene DapB, were stained with the other tissue section on the same slide. Minimal non-specific staining is detected in the negative control section in all three channels, which suggests the additional CBT amplification does not cause the nonspecific staining but only enhances the signal of the target mRNA (FIG. 8). This indicates that the new platform can effectively detect diverse mRNAs across a wide range of expression levels in FFPE tissue, with minimal non-specific staining.

### Efficiency of Deactivation of Horseradish Peroxidase and Biotin Removal.

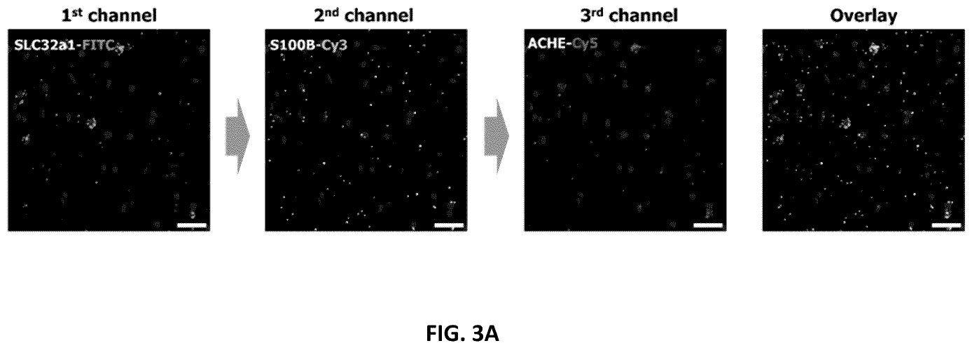

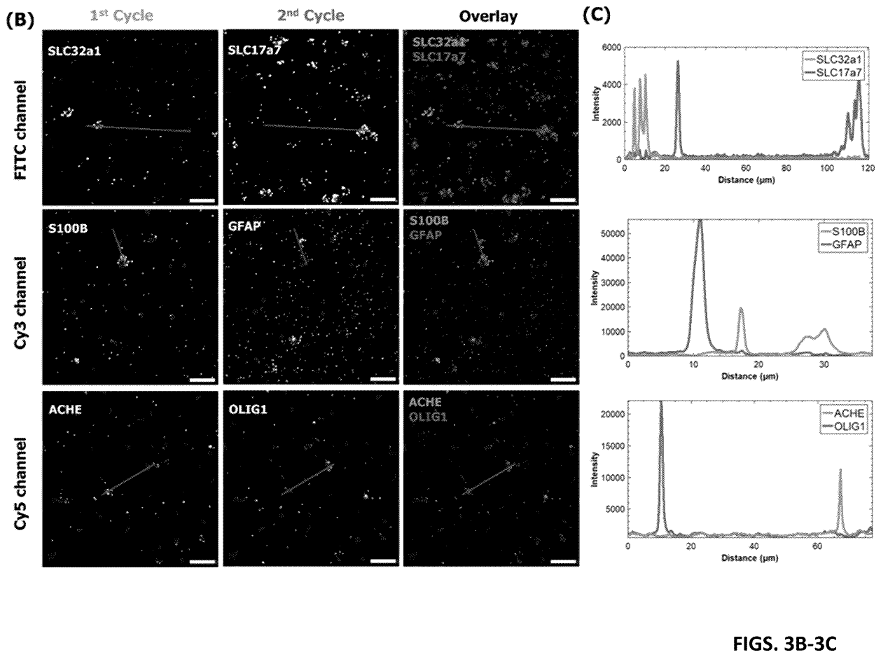

With the inclusion of the additional CBT signal amplification step in this new approach, another critical consideration for achieving multiplexing is ensuring that the deposited CBT and SA-HRP do not produce crosstalk signals in the subsequent cycle staining. Two key factors must be addressed: first, unbound biotin may bind to the subsequent SA-HRP, and second, incomplete deactivation of HRP could lead to the repeating deposition of CBT and CFT in the next cycle. Ideally, the deposited CBT should primarily bind to SA-HRP, while the HRP blocker efficiently deactivates the deposited SA-HRP and channel HRP. Additionally, between cycles, the TCEP solution can assist in removing unbound biotin from the remaining CBT and assist the deactivation of HRP. Furthermore, excess free biotin is used for streptavidin blocking, which can prevent the nonspecific binding of CBT in the following staining. To evaluate the crosstalk issue between each channel, we selected three neural markers (SLC32a1, S100B, and ACHE) and stained them sequentially with three different CFTs (tyramide-N₃-FITC, tyramide-N₃-Cy3, and tyramide-N₃-Cy5). Nonoverlapping and distinct staining patterns observed in all three channels demonstrate that the deposited CBT and SA-HRP do not interfere with subsequent channel staining (FIG. 3A).

Another criterion for the success of this RNA profiling method is the efficient removal of fluorophores and probe stripping. Lu et al. [24] demonstrated that target and amplification probes can be efficiently removed using DNase I and high-concentration formamide. Based on this, we employed a similar probe-stripping protocol to remove the hybridized target and amplification probes. Besides, the three CFTs used in this study can be chemically removed using TCEP and PTA solutions. To validate the efficiency of fluorophore removal and probe stripping, six mRNA probes (Cycle 1: SLC32a1-FITC, S100B-Cy3, and ACHE-Cy5; Cycle 2: SLC17a7-FITC, GFAP-Cy3, and Olig1-Cy5) were sequentially stained with three fluorophores over two iterative cycles, interspersed with TCEP and PTA treatment for fluorophore removal and complete probe stripping. The reiterative staining results are shown in FIGS. 3B and 3C. In all three channels, the staining signals from the first cycle (shown in green, left panels in FIG. 3B) were completely removed after HRP deactivation, fluorophore cleavage, and probe stripping. In the subsequent cycle, the new staining (shown in magenta, right panels in FIG. 3B) displayed distinct patterns compared to the previous cycle. This confirms the efficiency of HRP deactivation, removal of deposited biotin and fluorophores, and probe stripping processes. The results suggest minimal crosstalk between cycles, enabling successful multiplexed staining.

### Multiplexed In Situ RNA Profiling in Human Cerebral Cortex FFPE Tissue

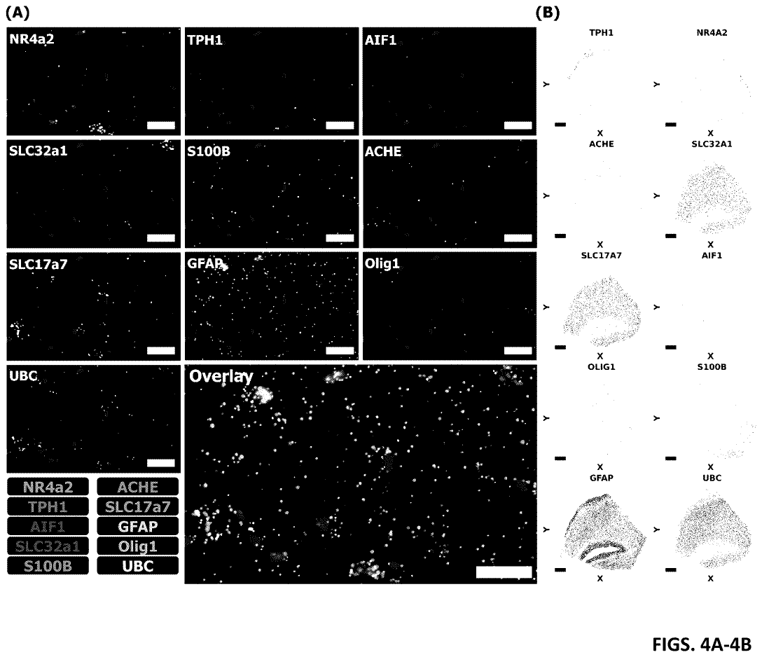

To demonstrate the feasibility of our new approach for multiplexed RNA profiling in human brain FFPE tissue, we stained nine neural mRNA targets and one housekeeping gene in human cerebral cortex tissue (FIG. 4A). Through multiple iterative cycles of target staining, fluorescence imaging, HRP deactivation, fluorophore removal, and probe stripping, we successfully identified NR4A2, TPH1, AIF1, SLC32A1, S100B, ACHE, SLC17A7, GFAP, OLIG1, and UBC within the same tissue sample. The staining details and associated target cell types for each transcript are provided in Table 1. Processed whole-tissue staining results for the ten transcripts are shown in FIG. 4B. Distinct staining patterns were observed, reflecting varied expression levels of these neural mRNAs. These spatially resolved and multiplexed transcriptomic profiles enable further analysis of cellular heterogeneity and spatial organization within the human cerebral cortex.

Unique Neural Cell Types and their Spatial Distribution in Human Cerebral Cortex Tissue

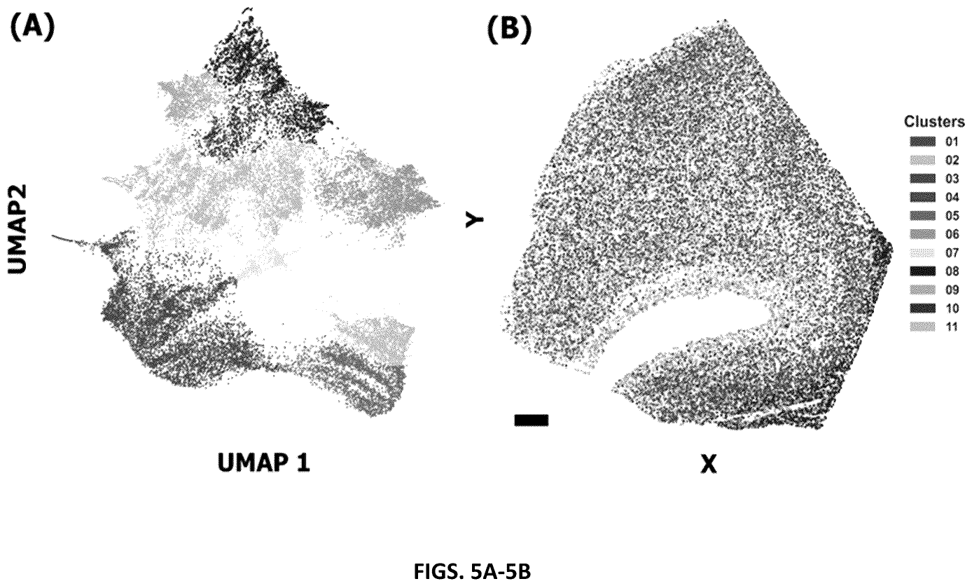

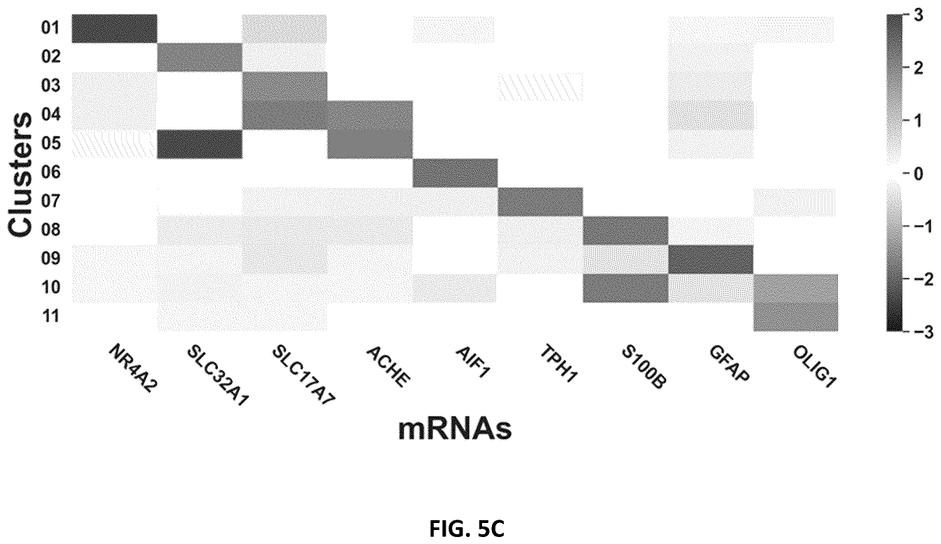

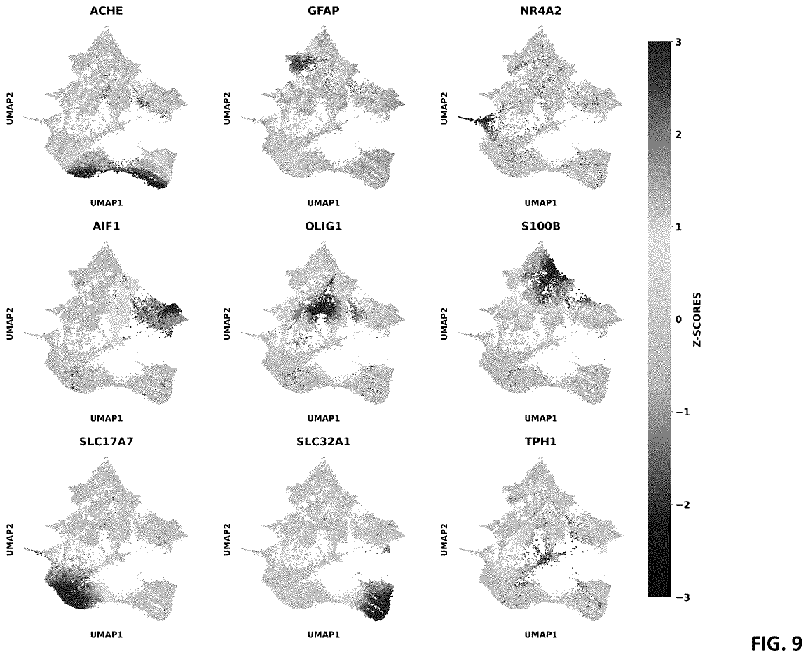

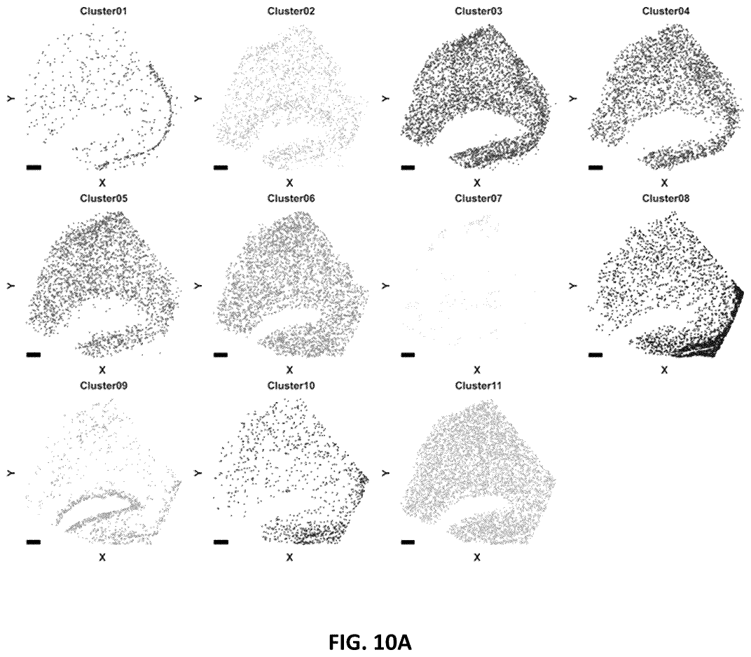

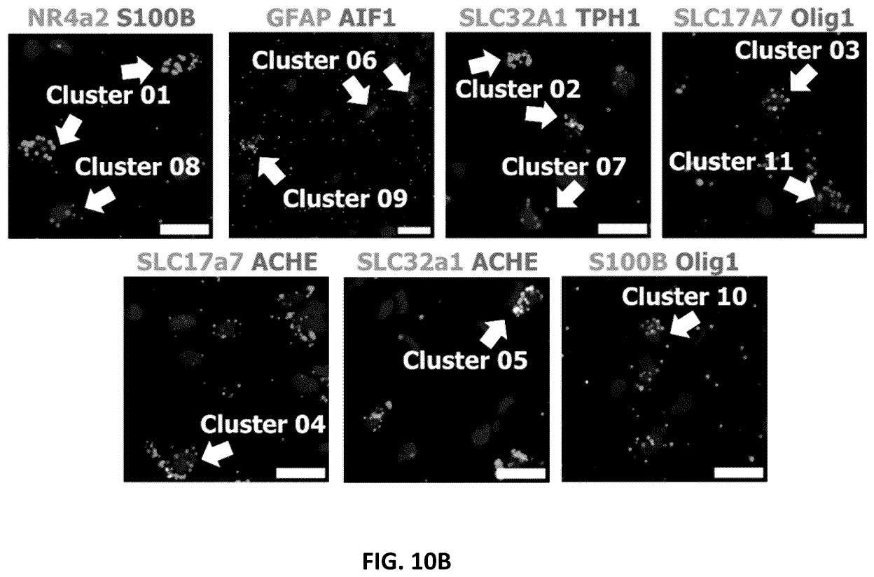

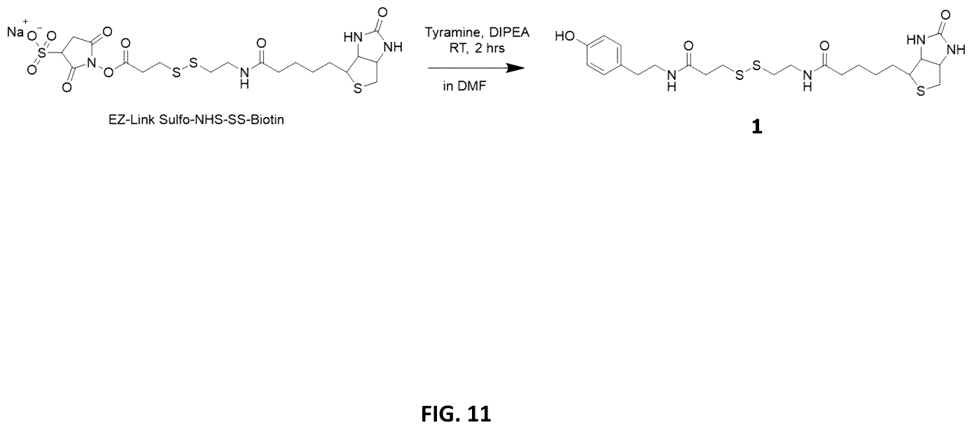

To generate transcriptomic profiles, spot counts per cell, along with their spatial coordinates for 26,000 cells were collected using the image analysis software CellProfiler [27]. This digitized single-cell in situ multiplexed RNA profiles enabled us to further investigate the composition of neural cell types based on transcript abundance. Additionally, we analyzed the spatial relationships between cells by evaluating the ratio of neighboring cell types within the human cerebral cortex. To visualize the similarity among 26,000 cells based on the nine neural mRNA profiles, their expression levels were analyzed using the dimensionality reduction algorithm Uniform Manifold Approximation and Projection (UMAP) [30]. The Z-normalized expression levels of the nine neural markers are displayed on a 2-dimensional UMAP plot (FIG. 9). Following the generation of optimized UMAP results from the nine neural expression profiles, an unsupervised Leiden clustering algorithm was applied to partition the cells into eleven distinct clusters (FIG. 5A). In FIG. 5B, the eleven annotated clusters are mapped back to their original tissue locations, revealing distinct staining patterns. The distribution of individual clusters within the tissue is provided in FIG. 10A.

By analyzing the mRNA expression level composition of these assigned 11 clusters (FIG. 5C), we found that most clusters were predominantly characterized by a single mRNA, corresponding to specific neural markers. The target cell types of these nine neuron marks are listed in Table 1. For example, the cells classified into cluster 1 exhibit enrichment in mRNA NR4A2, suggesting that cells annotated in cluster 1 likely represented dopaminergic neurons, which show a tail-like staining pattern in the cerebral cortex (red cluster in FIG. 5B). Besides, two astrocytic markers, GFAP and S100B, are enriched in two different regions in this tissue (orange and blue clusters in FIG. 5B). The difference in staining patterns might be due to the subpopulation of astrocytes. The S100B has been reported to be selectively expressed in protoplasmic astrocytes in gray matter [32], while the GFAP is not only expressed in protoplasmic astrocytes but also fibrous astrocytes in white matter [33]. Moreover, there are three clusters, clusters 4 (enriched in ACHE & SLC17a7), cluster 5 (enriched in ACHE & SLC32a1), and cluster 10 (enriched in S100B & Olig1), that indicate the cells with double-enriched mRNA profiles might be different sub-cell types compared to the cells which only enriched in single mRNA. For example, S100B has been reported to be essential in the differentiation and maturation of oligodendrocytes [32, 34]. This might suggest the cells co-expressing both Olig1 and S100B are the oligodendrocyte precursor cells (OPCs), while the cells expressing only mRNA marker Olig1 (cluster 11) should be the mature oligodendrocytes. Besides, SLC17A7 (VGLUT1) and SLC32A1 (VGAT) have been reported as reliable markers for identifying glutamatergic neurons [35] and GABAergic neurons [36], respectively. In our results, we found these two mRNAs, SLC32A1 and SLC17A7, are not only specifically enriched in clusters 2 and 3, respectively, but also co-expressed with another mRNA ACHE (Acetylcholinesterase) in clusters 5 and 4 (FIG. 5C). The ACHE, involved in acetylcholine (ACh) degradation, is a marker of cholinergic neurons. The co-transmission of GABA [37, 38] and glutamate from cholinergic neurons have been reported. These different transmitter expression patterns might indicate specific synaptic communication. To ensure the assigned clusters are not artifacts, the fluorescence staining of annotated 11 clusters is shown in FIG. 10B. The green and magenta spots show the corresponding mRNA staining.

### Distinct Composition of Neural Cells in the Human Cerebral Cortex

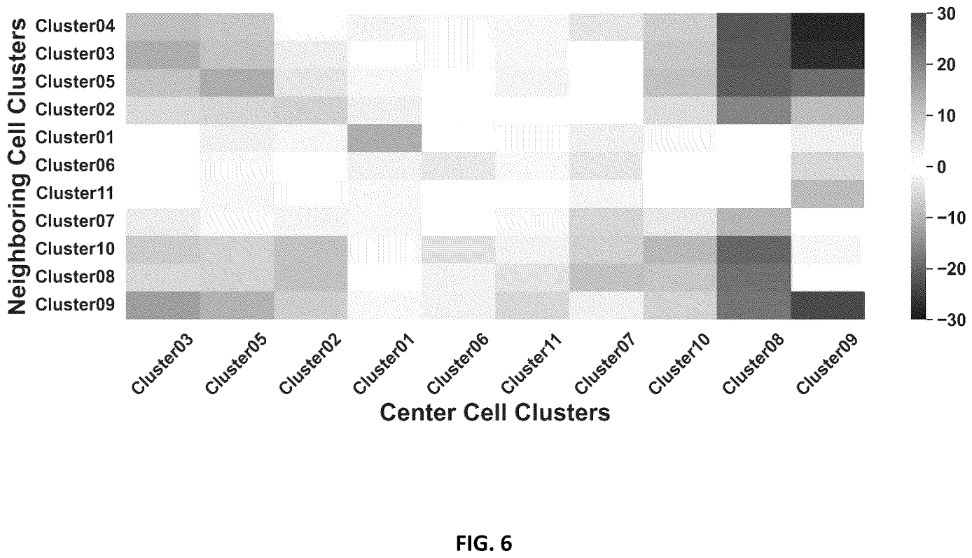

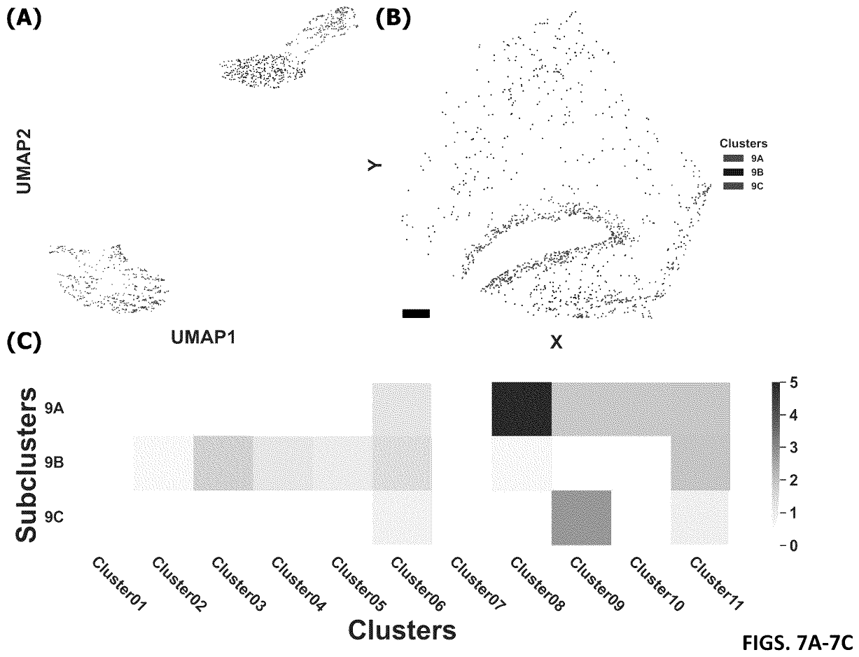

After defining the neural clusters based on RNA expression levels, we further explored potential subclusters within these major clusters by examining their spatial environments. Neighbor analysis was conducted by calculating the odds of annotated clusters appearing within a 75 μm radius of selected center cells. The projected odds values for each cluster are visualized in a heatmap (FIG. 6). A higher odds value of the neighbor cluster cell indicates a greater likelihood of proximity to the center cell. The neighbor analysis code used in this study was developed by Goltsev et al. [40]. We observed that neuronal clusters 2, 3, 4, and 5, which express different neurotransmitters, frequently associate with one another. This pattern may suggest the presence of potential neuronal networks. Additionally, the astrocytic clusters (clusters 8 and 9) tend to co-occur in proximity, likely due to the enriched expression of genes associated with astrocyte-specific markers. The major clusters can be further subdivided into subclusters by analyzing the composition of their neighboring cells. Here, we present subcluster analysis for cluster 9, which is enriched in GFAP mRNA. Cells within a 75 μm radius of cluster 9 cells were counted to generate a new “cell neighborhood” profile, consisting of various assigned clusters. This neighborhood profile was then subjected to UMAP analysis and Leiden clustering to identify unique subclusters. Three subclusters (09A-09C) with unique compositions of neural cell types were derived from cluster 9. (FIG. 7A and FIG. 7C). By mapping back the subcluster in the original position in the tissue (FIG. 7B), we can observe that subclusters 09A and 09C are predominantly located in the regions of cluster08 (S100B enriched cells) and cluster09 (GFAP enriched cells), respectively. The cells of subcluster 09B are not located in these astrocytic regions. These spatially defined subclusters may offer new insights into the microenvironments of astrocytes, revealing specific cell type compositions in different tissue locations.

### CONCLUSIONS

In summary, we established an ultra-sensitive platform for multiplexed single-cell in situ RNA analysis with RNAscope® fluorescent v2 kit, cleavable biotin tyramide, and cleavable fluorescent tyramides. With this enhanced signal amplification, we significantly improved the detection of green (FITC) and orange (Cy3) channels in highly autofluorescent tissues, offering greater flexibility for detecting low-abundance mRNAs in FFPE samples. To demonstrate the feasibility of this approach, we successfully performed multiplexed staining of ten different mRNA targets in human cerebral cortex FFPE tissue. The results have showcased extraordinary sensitivity in detecting varied abundance of mRNAs unambiguously. Furthermore, eleven neural-related clusters were identified based on the expression profiles of nine neural mRNAs. By integrating spatial context information, unique subclusters within each neural cluster were further defined, offering deeper insights into the microenvironment of individual cells in human brain tissue. This highly sensitive RNA imaging platform enables comprehensive multiplexed RNA profiling, providing a powerful tool for uncovering complex biological events.

Besides, our advanced RNA-FISH technique, which integrates strong signal amplification with high multiplexing capabilities, offers a promising approach for detecting diverse short RNA fragments in FFPE tissues. By reducing the number of target probes required while maintaining high sensitivity, this method has the potential to be applied in detecting various RNA species relevant to biology and disease. This approach may enable the differentiation of mRNA splice variants[41] and circular RNAs[42] by detecting specific exon-exon junctions, which could be valuable for studying their roles in gene regulation and disease progression. Additionally, the technique holds promise for detecting microRNAs[43], which are key regulators of gene expression and are implicated in various diseases, including cancer and cardiovascular disorders. Our method may also be applicable for evaluating gene-editing outcomes, such as detecting CRISPR-mediated transcript modifications[44], which is crucial for assessing gene-editing efficiency and specificity. Furthermore, it has the potential to be used in gene therapy research, including the detection of AAV-mediated transgene expression[45], providing insights into therapeutic delivery and efficacy. Overall, these capabilities highlight the potential of our RNA-FISH platform to support a wide range of applications in gene regulation research, disease pathology, and gene therapy development.

### REFERENCES

1. Williams, C. G., et al., An introduction to spatial transcriptomics for biomedical research. Genome Medicine, 2022. 14(1): p. 68.
2. Metzker, M. L., Sequencing technologies—the next generation. Nature Reviews Genetics, 2010. 11(1): p. 31-46.
3. Hoheisel, J. D., Microarray technology: beyond transcript profiling and genotype analysis. Nature Reviews Genetics, 2006. 7(3): p. 200-210.
4. Huang, K. and A.A. Martí, Recent trends in molecular beacon design and applications. Analytical and Bioanalytical Chemistry, 2012. 402(10): p. 3091-3102.
5. Guo, J., J. Ju, and N. J. Turro, Fluorescent hybridization probes for nucleic acid detection. Anal Bioanal Chem, 2012. 402(10): p. 3115-25.
6. Raj, A., et al., Imaging individual mRNA molecules using multiple singly labeled probes. Nature Methods, 2008. 5(10): p. 877-879.
7. Guo, J., et al., Four-color DNA sequencing with 3′-O-modified nucleotide reversible terminators and chemically cleavable fluorescent dideoxynucleotides. Proceedings of the National Academy of Sciences, 2008. 105(27): p. 9145-9150.
8. Martí, A. A., et al., Fluorescent Hybridization Probes for Sensitive and Selective DNA and RNA Detection. Accounts of Chemical Research, 2007. 40(6): p. 402-409.
9. Shaffer, S. M., et al., Rare cell variability and drug-induced reprogramming as a mode of cancer drug resistance. Nature, 2017. 546(7658): p. 431-435.
10. Xiao, L. and J. Guo, Multiplexed single-cell in situ RNA analysis by reiterative hybridization. Analytical Methods, 2015. 7(17): p. 7290-7295.
11. Xiao, L. and J. Guo, Single-Cell in Situ RNA Analysis With Switchable Fluorescent Oligonucleotides. Frontiers in Cell and Developmental Biology, 2018. 6.
12. Xiao, L., R. Liao, and J. Guo, Highly Multiplexed Single-Cell In Situ RNA and DNA Analysis by Consecutive Hybridization. Molecules, 2020. 25(21): p. 4900.
13. Mondal, M., et al., Highly multiplexed single-cell in situ RNA and DNA analysis with bioorthogonal cleavable fluorescent oligonucleotides. Chemical Science, 2018. 9(11): p. 2909-2917.
14. Xia, C., et al., Multiplexed detection of RNA using MERFISH and branched DNA amplification. Scientific Reports, 2019. 9(1): p. 7721.
15. Moffitt, J. R., et al., Molecular, spatial, and functional single-cell profiling of the hypothalamic preoptic region. Science, 2018. 362(6416).
16. Moffitt, J. R., et al., High-throughput single-cell gene-expression profiling with multiplexed error-robust fluorescence in situ hybridization. Proceedings of the National Academy of Sciences, 2016. 113(39): p. 11046-11051.
17. Chen, K. H., et al., RNA imaging. Spatially resolved, highly multiplexed RNA profiling in single cells. Science, 2015. 348(6233): p. aaa6090.
18. Eng, C.-H. L., et al., Transcriptome-scale super-resolved imaging in tissues by RNA seqFISH+. Nature, 2019. 568(7751): p. 235-239.
19. Lubeck, E., et al., Single-cell in situ RNA profiling by sequential hybridization. Nature Methods, 2014. 11(4): p. 360-361.
20. Lee, J. H., et al., Fluorescent in situ sequencing (FISSEQ) of RNA for gene expression profiling in intact cells and tissues. Nature Protocols, 2015. 10(3): p. 442-458.
21. Ke, R., et al., In situ sequencing for RNA analysis in preserved tissue and cells. Nature Methods, 2013. 10(9): p. 857-860.
22. Erben, T., et al., What to do with high autofluorescence background in pancreatic tissues—an efficient Sudan black B quenching method for specific immunofluorescence labelling. Histopathology, 2016. 69(3): p. 406-422.
23. Blow, N., Tissue issues. Nature, 2007. 448(7156): p. 959-960.
24. Xiao, L., J. Labaer, and J. Guo, Highly Sensitive and Multiplexed In Situ RNA Profiling with Cleavable Fluorescent Tyramide. Cells, 2021. 10(6): p. 1277.
25. Wang, F., et al., RNAscope: a novel in situ RNA analysis platform for formalin-fixed, paraffin-embedded tissue. The Journal of Molecular Diagnostics, 2012. 14(1): p. 22-29.
26. Schindelin, J., et al., Fiji: an open-source platform for biological-image analysis. Nature Methods, 2012. 9(7): p. 676-682.
27. Carpenter, A. E., et al., CellProfiler: image analysis software for identifying and quantifying cell phenotypes. Genome Biology, 2006. 7(10): p. R100.
28. Bankhead, P., et al., QuPath: Open source software for digital pathology image analysis. Scientific Reports, 2017. 7(1): p. 16878.
29. Wolf, F. A., P. Angerer, and F.J. Theis, SCANPY: large-scale single-cell gene expression data analysis. Genome Biology, 2018. 19(1): p. 15.
30. McInnes, L., J. Healy, and J. Melville, Umap: Uniform manifold approximation and projection for dimension reduction. arXiv preprint arXiv:1802.03426, 2018.
31. Traag, V. A., L. Waltman, and N. J. van Eck, From Louvain to Leiden: guaranteeing well-connected communities. Scientific Reports, 2019. 9(1): p. 5233.
32. Du, J., et al., S100B is selectively expressed by gray matter protoplasmic astrocytes and myelinating oligodendrocytes in the developing CNS. Molecular Brain, 2021. 14(1).
33. Tabata, H., Diverse subtypes of astrocytes and their development during corticogenesis. Front Neurosci, 2015. 9: p. 114.
34. Santos, G., et al., Impaired oligodendrogenesis and myelination by elevated S100B levels during neurodevelopment. Neuropharmacology, 2018. 129: p. 69-83.
35. Fremeau, R. T., et al., VGLUTs define subsets of excitatory neurons and suggest novel roles for glutamate. Trends in Neurosciences, 2004. 27(2): p. 98-103.
36. Wojcik, S. M., et al., A Shared Vesicular Carrier Allows Synaptic Corelease of GABA and Glycine. Neuron, 2006. 50(4): p. 575-587.
37. Granger, A. J., et al., Developmental regulation of GABAergic gene expression in forebrain cholinergic neurons. Front Neural Circuits, 2023. 17: p. 1125071.
38. Granger, A. J., et al., Cotransmission of acetylcholine and GABA. Neuropharmacology, 2016. 100: p. 40-46.
39. Trudeau, L.E. and S. El Mestikawy, Glutamate Cotransmission in Cholinergic, GABAergic and Monoamine Systems: Contrasts and Commonalities. Front Neural Circuits, 2018. 12: p. 113.
40. Goltsev, Y., et al., Deep Profiling of Mouse Splenic Architecture with CODEX Multiplexed Imaging. Cell, 2018. 174(4): p. 968-981.e15.
41. Rogalska, M. E., C. Vivori, and J. Valcárcel, Regulation of pre-mRNA splicing: roles in physiology and disease, and therapeutic prospects. Nature Reviews Genetics, 2023. 24(4): p. 251-269.
42. Zhang, Z., T. Yang, and J. Xiao, Circular RNAs: Promising Biomarkers for Human Diseases. EBioMedicine, 2018. 34: p. 267-274.
43. Rupaimoole, R. and F.J. Slack, MicroRNA therapeutics: towards a new era for the management of cancer and other diseases. Nature Reviews Drug Discovery, 2017. 16(3): p. 203-222.
44. Uddin, F., C.M. Rudin, and T. Sen, CRISPR Gene Therapy: Applications, Limitations, and Implications for the Future. Front Oncol, 2020. 10: p. 1387.
45. Wang, D., P.W.L. Tai, and G. Gao, Adeno-associated virus vector as a platform for gene therapy delivery. Nature Reviews Drug Discovery, 2019. 18(5): p. 358-378.

### Example 2

Alternative splicing affects almost all the genes with multiple exons in mammals¹. And the number of transcription start sites is one order of magnitude higher than the number of genes². Additionally, at least half of all genes have alternative polyA-sites³. Taken together, these vast abundances of alternative sites lead to a huge number of varied RNA isoforms, which regulate normal physiology and malfunction in diseases⁴. How the combined status of different RNA isoforms in individual cells are regulated, how do they affect cell type classification, and what is the 3-D organization of these diverse cell types in tissues are largely unknown. Methods enabling comprehensive single-cell spatial RNA isoforms analysis are critically needed to answer these important questions.

As the most common type of preserved clinical samples, formalin-fixed paraffin-embedded (FFPE) tissues are extensively used for routine diagnosis and studies of disease mechanisms⁵. However, very few protein isoform specific antibodies are available for immunohistochemistry. Clearly, highly multiplexed RNA isoform imaging in FFPE tissues is critically needed to unlock major mysteries in neuroscience, cancer and stem cell biology⁶. It will also enhance the diagnosis accuracy and advance our understanding of disease pathogenesis.

Recently, several technologies have been developed for spatial transcriptomics analysis, such as Nanostring GeoMx⁷, Slide-seq (10× genomics Visium)⁸, DBiT-seq⁹ and SiT¹⁰ etc. However, the spatial resolution of these approaches is ~10 μm. As a result, the averaged results from 3-10 cells are generated in each analysis pixel (Table 2).

TABLE 2
Spatial transcriptomics technologies

| | | | | Transcript count |
|---|---|---|---|---|
| | Single cell | Isoforms | Multiplexed | per gene per cell in |
| | analysis | analysis | analysis | FFPE tissues |
| GeoMx, Visium, DBiT-seq, | No | Yes | Yes | no single cell |
| SiT | | | | resolution |
| Basescope | Yes | Yes | No | 5-20 |
| MERFISH, SeqFISH, | Yes | No | Yes | <0.25 |
| SABER FISH, STARmap, | | | | |
| in situ sequencing, Xenium, | | | | |
| CosMx, Visium HD | | | | |
| Proximity labeling probes | Yes | Yes | Yes | 5-20 |

To enable spatial transcriptomics analysis at the subcellular resolution, several laboratories including ours have explored different multiplexed RNA fluorescence in situ hybridization (FISH) approaches. However, none of the existing methods allow highly multiplexed single-cell RNA isoforms analysis in FFPE tissues¹¹, due to two fundamental limitations (Table 2). (i) Low multiplexing capacity: the current RNA isoform imaging method, Basescope¹², can only visualize up to two RNA species in one specimen. As chromophores rather than fluorophores are used in Basescope to overcome the autofluorescence background, its multiplexing capacity is limited. (ii) Low sensitivity: other highly multiplexed RNA profiling technologies can only detect <0.25 transcript for each gene per cell in FFPE tissues^{13,14}, or have not demonstrated applicability in FFPE tissues. These technologies include MERFISH¹⁵, Seq-FISH¹⁶, SABER FISH¹⁷, in situ sequencing¹⁸, STARmap¹⁹, 10× genomics Xenium²⁰, Visium HD²¹, and Nanostring CosMx²². Almost all of these methods apply branched DNA structures (20×20×20=8,000 fluorophores)¹² or rolling circle amplification¹⁹ to amplify the signals. As a result, only <10⁴ fluorophores are labeled on each target hybridization probe. However, as shown in our publication²³, even with ~10⁶ fluorophores per probe deposited by RNAscope (branched DNA and horseradish peroxidase (HRP) amply signals 10⁴ and 10² times, respectively^{12,24}), it is still challenging to detect low-abundance RNA in highly fluorescent FFPE tissues, especially in the green and yellow fluorescence channels. Additionally, due to their limited sensitivity, almost all the current multiplexed RNA imaging methods require >20 probes hybridized to the same target. Thus, they can only detect transcripts longer than 500 bases. As a result, the existing multiplexed RNA imaging methods are unable to profile RNA isoforms in FFPE tissues.

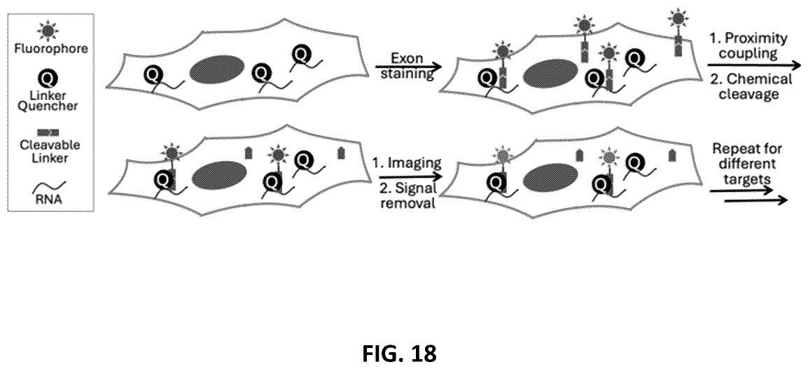

Our objective is to develop a multiplexed RNA isoform imaging approach, which enables comprehensive single-cell RNA isoform profiling in FFPE tissues in situ. In this approach (FIG. 18), RNA targets in FFPE tissues are first labeled with quencher molecules by an approach similar to the gold standard RNAscope. In brief, the RNA is hybridized by ~20 pairs of target-specific oligonucleotide probes. Only when those two independent probes in each pair hybridize to the targets in tandem will the subsequent signal amplification be initiated. In this way, the staining specificity is ensured. Afterwards, the staining signals are amplified by both branched DNA and enzymatic tyramide signal amplification (TSA) using HRP, to deposit quencher molecules. Then after probe stripping, a given exon on the same RNA is stained similarly using cleavable fluorophores. As the short exon can only be hybridized by 1-3 pairs of oligonucleotide probes, additional rounds of TSA will be applied to further amplify the signal, so that all the individual exons with various lengths can be sensitively detected. To remove the over-amplified nonspecific signal, a proximity coupling reaction between the quencher and the cleavable linker is carried out. Since HRP only deposits molecules within 20 nm of the target^{25,26}, only the cleavable linker in close proximity to the RNA will be deactivated. And following chemical cleavage, all the nonspecific signals will be erased, while the real exon staining signal will remain intact. After image capture to quantify the identities, positions and copy numbers of the stained RNA isoforms, all the fluorescence signals are photobleached and the oligonucleotide probes are stripped off, to initiate the next cycle. Through reiterative cycles of staining, imaging, probe stripping and signal removal, comprehensive RNA isoform analysis in FFPE tissues can be achieved in single cells in situ. This novel proximity labeling strategy, together with innovations in signal amplification methods, completely overcomes all the fundamental limitations of the current methods.

Distinguishing RNA isoforms from the same gene necessitates the precise staining of individual short exons. To achieve this in highly autofluorescent FFPE tissues, extremely efficient signal amplification is required. However, all the existing multiplexed RNA imaging methods only have <10⁴ fluorophores on each target hybridization probe. In our approach, varied signal amplification methods are integrated to ensure the desired sensitivity. For example, branched DNA allows us to amplify the signal by 8,000 (20×20×20) times. And as one HRP molecule can enzymatically deposit up to 100 fluorophores²⁴, two additional rounds of HRP reactions will further amplify the signal by another 10⁴ (100×100) times. As a result, totally ~10⁸ (8,000×10⁴) fluorophores will be deposited per each target hybridization probe. By improving the transcript count of current methods from <0.25 to 5-20 per gene per cell (Table 2), our approach enables highly sensitive and multiplexed RNA isoform imaging in FFPE tissues.

With ~10⁸ times signal amplification, some non-specific signals will be inevitably generated. To maintain the high staining signal to noise ratio, only the cleavable linker within 20 nm of the target RNA will be deactivated by the pre-deposited quencher molecules. As a result, the following fluorophore cleavage reaction will only remove the non-specific staining background. And the real exon staining signals will not be erased. Thus, such proximity linker quenching reaction will dramatically enhance the signal to noise ratio. Consequently, our approach achieves high staining specificity and sensitivity at the same time.

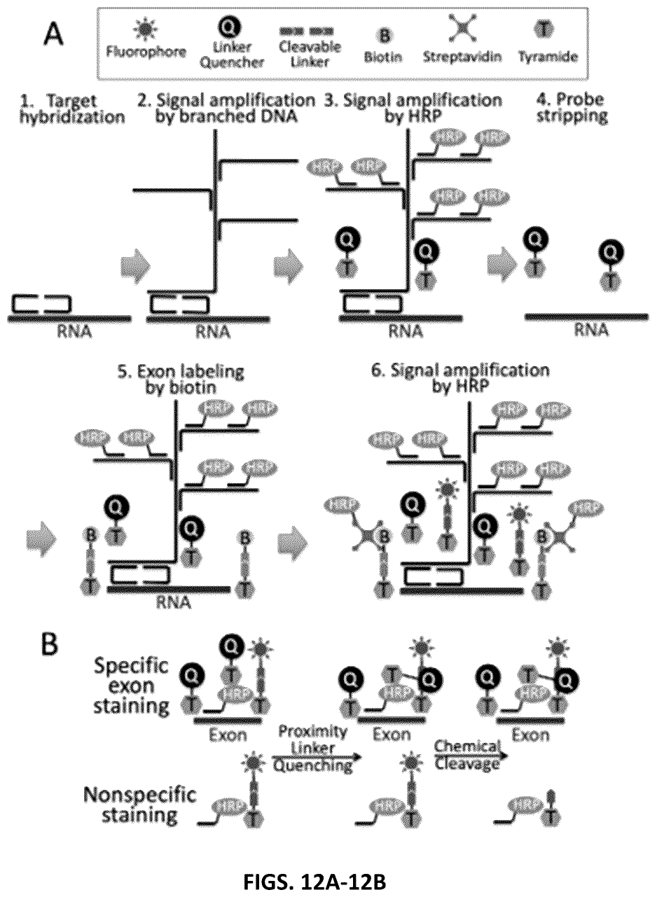

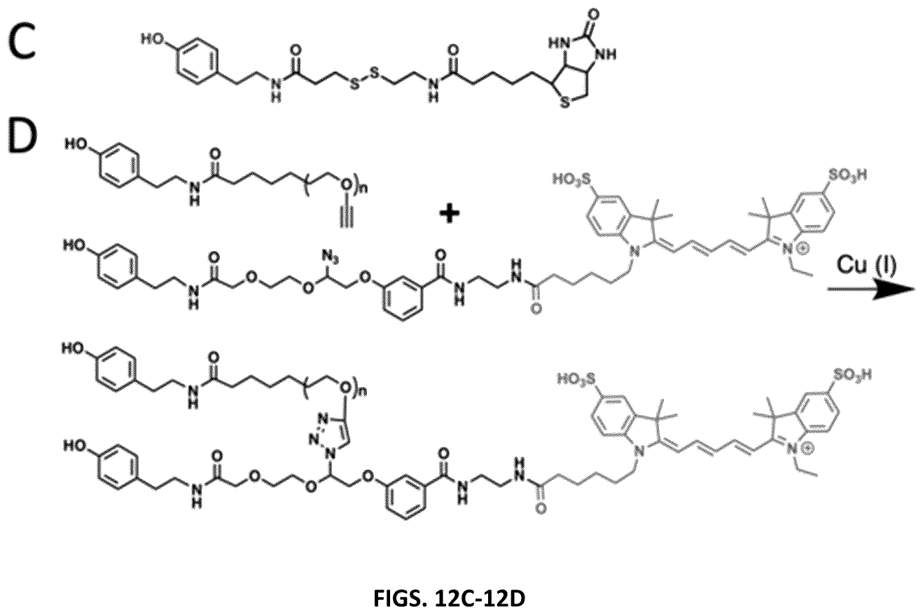

For multiplexed RNA isoform imaging in FFPE tissues, it requires individual exons to be detected, so that the varied RNA isoforms from the same gene can be differentiated. However, due to their limited sensitivity, the current multiplexed FISH technologies, such as MER-FISH¹⁵, Seq-FISH¹⁶, SABER FISH¹⁷, Nanostring CosMx²² and STARmap¹⁹ can't reliably profile short RNA sequences in highly autofluorescent FFPE tissues. To address this issue, we develop a multiplexed in situ RNA isoform profiling approach to enable the direct visualization of single exons down to 150 nucleotides. In this approach (FIG. 12A), transcripts are first hybridized by ~20 pairs of target-specific oligonucleotide probes (step 1). Only when these two independent probes in a pair hybridize to the target in tandem, the subsequent signal amplification by branched DNA will occur (step 2). Afterwards, horseradish peroxidase (HRP) is applied to further amplify the signal. Each HRP molecule can covalently deposit up to 100 alkyne tyramides, which functions as the quencher for the cleavable linker (step 3). After all the oligonucleotide probes are stripped by hot formamide (step 4), a specific exon of the RNA is hybridized by 3 pairs of oligonucleotide probes.

Branched DNA structures and HRP are applied again to amplify the signal and deposit biotin, which is tethered to tyramide through a disulfide based cleavable linker (FIG. 12C) (step 5). Each biotin will recruit a HRP conjugated streptavidin, which will then covalently deposit another ~100 cleavable fluorescent tyramides (step 6). Such branched DNA structures and two rounds of enzymatic signal amplification will enhance the signal intensities by ~10⁸ times. To ensure the staining specificity of highly sensitive exon imaging approach, we will apply click chemistry (FIG. 12B,D) to enable proximity labeling. Since the RNA target is first labeled by alkyne using an approach similar to the gold standard RNAscope, the staining specificity of the alkyne is ensured. And as HRP only deposits molecules within 20 nm of the target^{25,26}, only cleavable linkers in close proximity of the target RNA are deactivated by pre-deposited alkyne quenchers. As a result, the subsequent fluorophore cleavage reaction removes only the non-specific staining background distal to the RNA target, while leaving the true exon staining signals intact (FIG. 12B). This proximity-based linker quenching reaction significantly enhances the signal-to-noise ratio, enabling our approach to achieve both high staining specificity and sensitivity. After imaging, the fluorophores are photobleached, and the branched DNA is stripped off. Through cycles of target staining, imaging, signal removal and probe stripping, multiplexed RNA isoform imaging with high sensitivity and specificity can be achieved.

Various constructs of the paired target recognition probes, such as circular, double C, double Z and cruciform probes may be used with the amplification probe (FIG. 13).

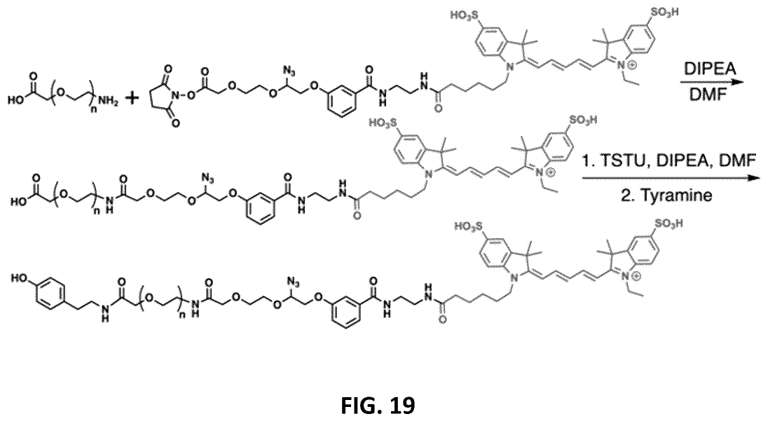

After alkyne and cleavable fluorescent tyramide are deposited, the click chemistry reaction is carried out with 0.6 mM CuSO4, 3 mM THPTA in PBS, and 4 mM of freshly made sodium ascorbate at room temperature for 30 minutes. Alkyne tyramide and cleavable fluorescent tyramide with poly(ethylene glycol) (PEG) linkers of 1-20 monomers are prepared by conjugating commercially available alkyne NHS ester with different PEG linkers to tyramine directly. To synthesize cleavable fluorescent tyramide, the PEG linker is coupled with cleavable fluorophore NHS ester similarly as we did recently in our publication²⁸. Then, the carboxyl group on the PEG linker will be activated and subsequently conjugated to tyramine (FIG. 19).

Methods for target staining and imaging are described in Example 1.

### Ultrasensitive Imaging of Individual Exons of ~40 Nucleotides in FFPE Tissues

The median size of human exons is only 120 base pairs, with 40% of all exons shorter than 100 base pairs³⁴. To visualize these short exons in FFPE tissues, we further improve the sensitivity of our approach to enable individual exon junctions or exons down to 40 base pairs to be detected. In this approach, the RNA targets are first stained with alkyne using ~20 pairs of target recognition probes, the same as in steps 1-4 in FIG. 12A. Subsequently, instead of using 3 pairs of probes, only 1 pair of target recognition probes is hybridized at the exon junction (FIG. 14). Then, a pair of amplification probes (Amp 1 probes) with multiple repeated sequences is hybridized to both target recognition probes. Only when both probes in Amp 1 are in close proximity will trigger the following signal amplification with branched DNA and enzymatic reactions. In this way, Amp 1 at the target site amplifies the signals, while the nonspecifically bound Amp 1 will not generate false positive signals. With repetitive sequences on Amp 1, the detection sensitivity of our approach can be improved by another order of magnitude, while maintaining the staining specificity. Through reiterative cycles of target staining, imaging, photobleaching and probe stripping, multiplexed exon junction imaging can be achieved in FFPE tissues.

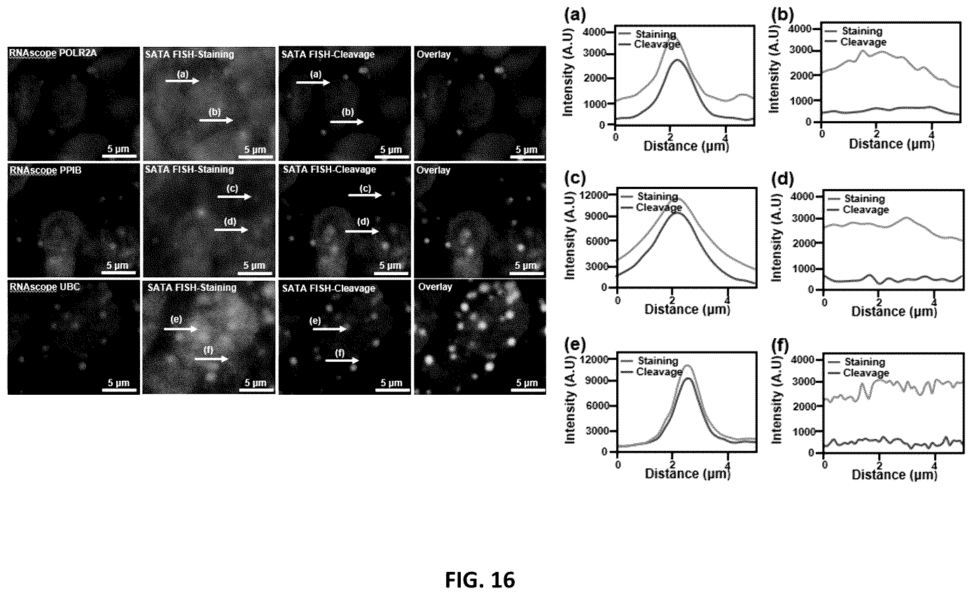

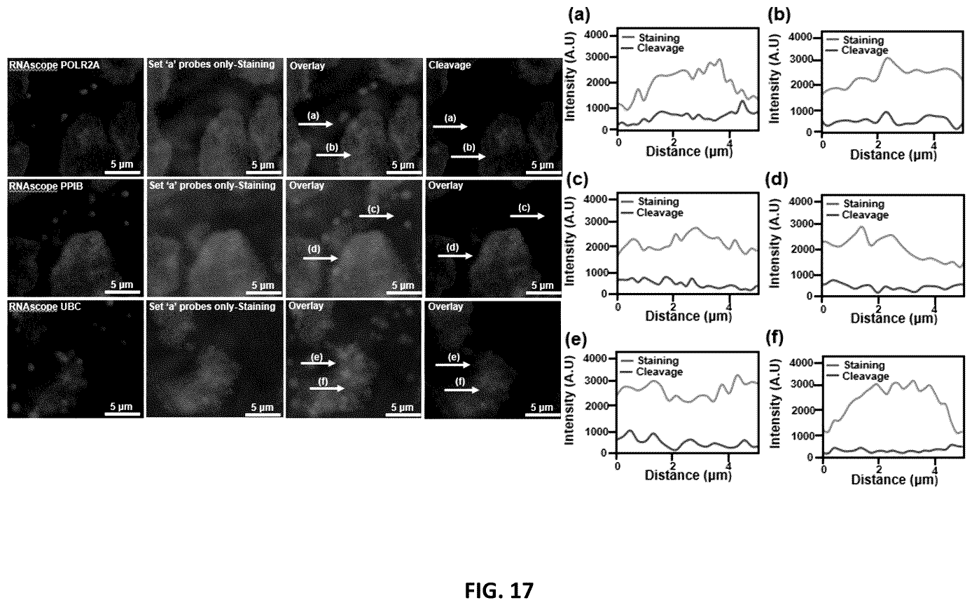

FIG. 16 shows a positive control of the staining and stain removal at specific staining sites using an amplification probe with a pair of target probes. FIG. 17 shows a negative control using an amplification probe with only one target probe.

The current RNA isoform imaging technology, Basescope, can only detect up to 2 targets in one specimen. To enable comprehensive RNA isoform profiling, we develop a highly multiplexed RNA isoform imaging approach in which all the RNA targets are first labeled with alkyne simultaneously, using the method described in steps 1~4 in FIG. 12A. Then, 5 different RNA exons or exon junctions are stained together using the method developed in (FIG. 12A) or (FIG. 14) using different colored fluorophores. After click reaction, chemical cleavage and imaging, all the fluorophores are photobleached and all the oligonucleotide probes are stripped. Through 10 reiterative analysis cycles and 5 targets profiled in each cycle, 50-plex RNA isoform imaging can be achieved in FFPE tissues.

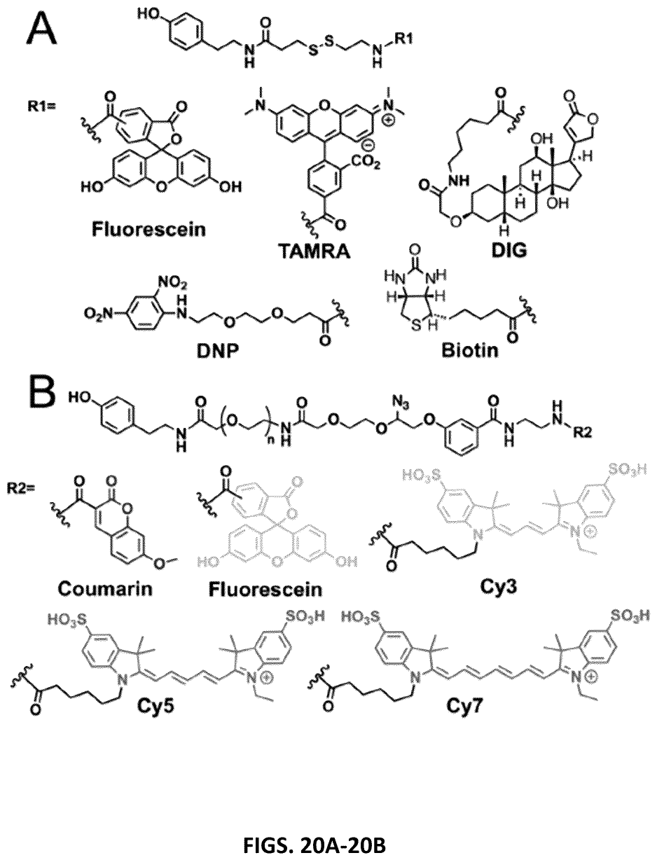

To increase the number of targets profiled in each cycle, 5 orthogonal branched DNA structures for signal amplification are designed. Five different cleavable hapten conjugated tyramide using fluorescein, tetramethylrhodamine (TAMRA), digoxigenin (DIG), dinitrophenol (DNP), and biotin are also designed (FIG. 20A). These cleavable hapten conjugated tyramide will be recognized by HRP labeled corresponding antibodies or streptavidin. In each cycle, all the 5 targets are first labeled simultaneously with the orthogonal branched DNA structures. Then the first target is stained with cleavable coumarin by 2 rounds of HRP signal amplification. After HRP deactivation with 0.2% H2O2, other targets are stained subsequently with cleavable Fluorescein, Cy3, Cy5, and Cy7 conjugated tyramide (FIG. 15B). In this way, 5 RNA targets can be stained in a day with an auto-stainer (Ventana), and 50-plex RNA isoform imaging can be completed in ~2 weeks. The detailed methods for probe preparation, target staining, click reaction, imaging, signal removal, imaging and data analysis are described above.

### REFERENCES EXAMPLE 2

1 Wang, E. T. et al. Alternative isoform regulation in human tissue transcriptomes. Nature 456, 470-476 (2008). https://doi.org/10.1038/nature07509
2 Consortium, F. et al. A promoter-level mammalian expression atlas. Nature 507, 462-470 (2014). https://doi.org/10.1038/nature13182
3 Tian, B., Pan, Z. & Lee, J. Y. Widespread mRNA polyadenylation events in introns indicate dynamic interplay between polyadenylation and splicing. Genome Res. 17, 156-165 (2007). https://doi.org/10.1101/gr.5532707
4 Joglekar, A. et al. A spatially resolved brain region- and cell type-specific isoform atlas of the postnatal mouse brain. Nat Commun 12, 463 (2021). https://doi.org/10.1038/s41467-020-20343-5
5 Blow, N. Tissue preparation: Tissue issues. Nature 448, 956-963 (2007). https://doi.org/doi: 10.1038/448959a.
6 Crosetto, N., Bienko, M. & Oudenaarden, A. V. Spatially resolved transcriptomics and beyond. Nature Reviews Genetics 16, 57-66 (2014). https://www.nature.com/articles/nrg3832
7 Asp, M., Bergenstrahle, J. & Lundeberg, J. Spatially Resolved Transcriptomes-Next Generation Tools for Tissue Exploration. Bioessays 42, e1900221 (2020). https://doi.org/10.1002/bies.201900221
8 Mirzazadeh, R. et al. Spatially resolved transcriptomic profiling of degraded and challenging fresh frozen samples. Nat Commun 14, 509 (2023). https://doi.org/10.1038/s41467-023-36071-5
9 Liu, Y. et al. High-Spatial-Resolution Multi-Omics Sequencing via Deterministic Barcoding in Tissue. Cell (2020). https://doi.org/10.1016/j.cell.2020.10.026
10 Lebrigand, K. et al. The spatial landscape of gene expression isoforms in tissue sections. Nucleic Acids Res. 51, e47 (2023). https://doi.org/10.1093/nar/gkad169
11 Wang, Y. S. & Guo, J. Multiplexed Single-Cell in situ RNA Profiling. Front Mol Biosci 8, 775410 (2021). https://doi.org/10.3389/fmolb.2021.775410
12 Wang, F. et al. RNAscope: A novel in situ RNA analysis platform for formalin-fixed, paraffin-embedded tissues. J. Mol. Diagn. 14, 22-29 (2012). https://doi.org/10.1016/j.jmoldx.2011.08.002
13 Cook, D. P. et al. A Comparative Analysis of Imaging-Based Spatial Transcriptomics Platforms. bioRxiv (2023). https://doi.org/10.1101/2023.12.13.571385
14 Wang, H. et al. Systematic benchmarking of imaging spatial transcriptomics platforms in FFPE tissues. bioRxiv (2023). https://doi.org/10.1101/2023.12.07.570603
15 Chen, K. H., Boettiger, a. N., Moffitt, J. R., Wang, S. & Zhuang, X. Spatially resolved, highly multiplexed RNA profiling in single cells. Science 1363, 1360-1363 (2015). https://doi.org/10.1126/science.aaa6090
16 Eng, C.-H. L. et al. Transcriptome-scale super-resolved imaging in tissues by RNA seqFISH+. Nature 568, 235-239 (2019). https://doi.org/10.1038/s41586-019-1049-y
17 Kishi, J. Y. et al. SABER amplifies FISH: enhanced multiplexed imaging of RNA and DNA in cells and tissues. Nat. Methods 16, 533-544 (2019). https://doi.org/10.1038/s41592-019-0404-0
18 Lee, J. H. et al. Highly multiplexed subcellular RNA sequencing in situ. Science (New York, N.Y.) 343, 1360-1363 (2014). https://doi.org/10.1126/science.1250212
19 Wang, X. et al. Three-dimensional intact-tissue sequencing of single-cell transcriptional states. Science 5691, 1-18 (2018). https://doi.org/10.1126/science.aat5691
20 Ke, R. et al. In situ sequencing for RNA analysis in preserved tissue and cells. Nat. Methods 10, 857-860 (2013). https://doi.org/10.1038/nmeth.2563
21 Janesick, A. et al. High resolution mapping of the tumor microenvironment using integrated single-cell, spatial and in situ analysis. Nature Communications 14 (2023). https://doi.org/10.1038/s41467-023-43458-x
22 He, S. et al. High-Plex Multiomic Analysis in FFPE Tissue at Single-Cellular and Subcellular Resolution by Spatial Molecular Imaging. bioRxiv, (2022). https://doi.org/10.1101/2021.11.03.467020
23 Xiao, L., Labaer, J. & Guo, J. Highly sensitive and multiplexed in situ RNA profiling with cleavable fluorescent tyramide. cells, 1277 (2021). https://doi.org/10.3390/cells10061277
24 Liao, R. et al. Highly Sensitive and Multiplexed Protein Imaging With Cleavable Fluorescent Tyramide Reveals Human Neuronal Heterogeneity. Frontiers in Cell and Developmental Biology 8, 614624 (2021). https://doi.org/10.3389/fcell.2020.614624
25 Martell, J. D. et al. A split horseradish peroxidase for the detection of intercellular protein-protein interactions and sensitive visualization of synapses. Nat. Biotechnol. 34, 774-780 (2016). https://doi.org/10.1038/nbt.3563
26 Martell, J. D. et al. Engineered ascorbate peroxidase as a genetically encoded reporter for electron microscopy. Nat. Biotechnol. 30, 1143-1148 (2012). https://doi.org/10.1038/nbt.2375
27 Xu, Q., Schlabach, M. R., Hannon, G. J. & Elledge, S. J. Design of 240,000 orthogonal 25mer DNA barcode probes. Proc. Natl. Acad. Sci. U.S.A. 106, 2289-2294 (2009). https://doi.org/10.1073/pnas.0812506106
28 Pham, T., Chen, Y., Labaer, J. & Guo, J. Ultrasensitive and Multiplexed Protein Imaging with Clickable and Cleavable Fluorophores. Anal. Chem. 96, 7281-7288 (2024). https://doi.org/10.1021/acs.analchem.4c01273
29 Mondal, M., Liao, R., Nazaroff, C. D., Samuel, A. D. & Guo, J. Highly multiplexed single-cell in situ RNA and DNA analysis with bioorthogonal cleavable fluorescent oligonucleotides. Chemical Science 9, 2909-2917 (2018). https://doi.org/10.1039/C7SC05089E
30 Xiao, L. & Guo, J. Single-Cell in Situ RNA Analysis With Switchable Fluorescent Oligonucleotides. Frontiers in Cell and Developmental Biology 6, 1-9 (2018). https://doi.org/10.3389/fcell.2018.00042
31 Xiao, L. & Guo, J. Multiplexed single-cell in situ RNA analysis by reiterative hybridization. Analytical Methods 7, 7290-7295 (2015). https://doi.org/10.1039/c5ay00500k
32 Xiao, L., Liao, R. & Guo, J. Highly Multiplexed Single-Cell In Situ RNA and DNA Analysis by Consecutive Hybridization. Molecules 25, 4900 (2020). https://doi.org/10.3390/molecules25214900
33 McQuin, C. et al. CellProfiler 3.0: Next-generation image processing for biology. PLoS Biol. 16, e2005970 (2018). https://doi.org/10.1371/journal.pbio.2005970
34 Mokry, M. et al. Accurate SNP and mutation detection by targeted custom microarray-based genomic enrichment of short-fragment sequencing libraries. Nucleic Acids Res. 38, e116 (2010). https://doi.org/10.1093/nar/gkq072
35 Sarter, M. & Parikh, V. Choline transporters, cholinergic transmission and cognition. Nature Reviews Neuroscience 6, 48-56 (2005). https://doi.org/10.1038/nrn1588
36 Hendricks, T. J. et al. Pet-1 ETS gene plays a critical role in 5-HT neuron development and is required for normal anxiety-like and aggressive behavior. Neuron 37, 233-247 (2003). https://www.cell.com/fulltext/S0896-6273(02)01167-4
37 Bráz, J. M. et al. Forebrain GABAergic neuron precursors integrate into adult spinal cord and reduce injury-induced neuropathic pain. Neuron 74, 663-675 (2012). https://doi.org/10.1016/j.neuron.2012.02.033
38 Baude, A., Strube, C., Tell, F. & Kessler, J.-P. Glutamatergic neurotransmission in the nucleus tractus solitarii: structural and functional characteristics. J. Chem. Neuroanat. 38, 145-153 (2009). https://doi.org/10.1016/j.jchemneu.2009.03.004
39 El-Husseini, A. E., Schnell, E., Chetkovich, D. M., Nicoll, R. A. & Bredt, D. S. PSD-95 involvement in maturation of excitatory synapses. Science (New York, N.Y.) 290, 1364-1368 (2000). https://www.science.org/doi/10.1126/science.290.5495.1364
40 Pozniak, C. D. et al. Sox 10 directs neural stem cells toward the oligodendrocyte lineage by decreasing Suppressor of Fused expression. Proceedings of the National Academy of Sciences of the United States of America 107, 21795-21800 (2010). https://doi.org/10.1073/pnas.1016485107
41 Marques, S. et al. Oligodendrocyte heterogeneity in the mouse juvenile and adult central nervous system. Science 352, 1326-1329 (2016). https://doi.org/10.1126/science.aaf6463
42 Kriegstein, A. & Alvarez-Buylla, A. The Glial Nature of Embryonic and Adult Neural Stem Cells. Annu. Rev. Neurosci. 32, 149-184 (2009). https://doi.org/10.1146/annurev.neuro.051508.135600
43 Heins, N. et al. Glial cells generate neurons: the role of the transcription factor Pax6. Nat. Neurosci. 5, 308-315 (2002). https://doi.org/10.1038/nn828
44 Jessen, K. R. & Mirsky, R. The origin and development of glial cells in peripheral nerves. Nature Reviews Neuroscience 6, 671-682 (2005). https://doi.org/10.1038/nrn1746
45 Bylund, M., Andersson, E., Novitch, B. G. & Muhr, J. Vertebrate neurogenesis is counteracted by Sox1-3 activity. Nat. Neurosci. 6, 1162-1168 (2003). https://doi.org/10.1038/nn1131

1. A method for multiplexed in situ analysis of nucleic acids in a tissue, the method comprising:
a) contacting the tissue with a target probe or a pair of target probes capable of hybridizing to a target nucleic acid;
b) contacting the tissue with a signal amplification molecule capable of binding to the target probe or pair of target probes, wherein the signal amplification molecule comprises a plurality of horseradish peroxidase (HRP) molecules;
c) contacting the tissue with a cleavable hapten tyramide (CHT) in a tyramide signal amplification (TSA) buffer;
d) contacting the tissue with HRP-conjugated streptavidin (SA-HRP);
e) contacting the tissue with a cleavable fluorophore tyramide (CFT) in a TSA buffer;
f) imaging the tissue;
g) contacting the tissue with an HRP deactivating agent;
h) contacting the tissue with biotin; and
i) repeating steps a) through h) for a plurality of rounds, wherein in each round, the target probe is capable of hybridizing to a different target nucleic acid; and wherein the fluorophore of each CFT is different than an immediately previous round.

2. The method of claim 1, further comprising:
j) contacting the tissue with a first cleavage solution comprising tris(2-carboxyethyl)phosphine (TCEP);
k) contacting the tissue with a second cleavage solution comprising 1,3,5-Triaza-7-phosphaadamantane (PTA);
l) contacting the tissue with DNase I;
m) contacting the tissue with a DNase I quenching buffer;
n) contacting the tissue with 70% formamide; and
o) repeating steps a) through n).

3. The method of claim 1, wherein step b) is done for about 30 minutes at about 40° C.

4. The method of claim 1, wherein step c) is done for about 30 minutes at about 40° C.

5. The method of claim 1, wherein between steps c) and d) the method further comprises washing the tissue in a buffer for about 2 minutes at about room temperature.

6. The method of claim 1, wherein step d) is done for about 30 minutes at about room temperature.

7. The method of claim 1, wherein steps d) and e) are done in an incubation buffer comprising 2×SSC, 1 mg/mL BSA, and 0.1% Tween-20.

8. The method of claim 1, wherein the target nucleic acid is an mRNA.

9. The method of claim 1, wherein the target nucleic acid comprises an exon-exon junction, and wherein the target probes are on opposite sides of the exon junction.

10. The method of claim 1, wherein the signal amplification molecule is hybridized to a plurality of branch probes; wherein each branch probe is hybridized to a plurality of HRP probes; and wherein each HRP probe is conjugated to an HRP molecule.

11. The method of claim 1, wherein in step i), the detectable fluorophore of each CFT is different.

12. The method of claim 11, wherein each of the detectable fluorophores of the CFT is selected from Cy3, Cy5, Cy7, CBT, and fluorescein.

13. The method of claim 1, wherein the hapten of the CHT is selected from biotin, fluorescein, TAMRA, DNP, and DIG.

14. The method of claim 1, wherein the tissue is a formalin-fixed paraffin-embedded tissue.

15. A cleavable hapten tyramide (CHT) comprising formula I) 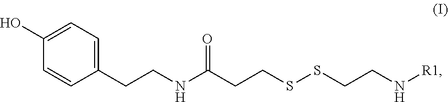
wherein R1 is

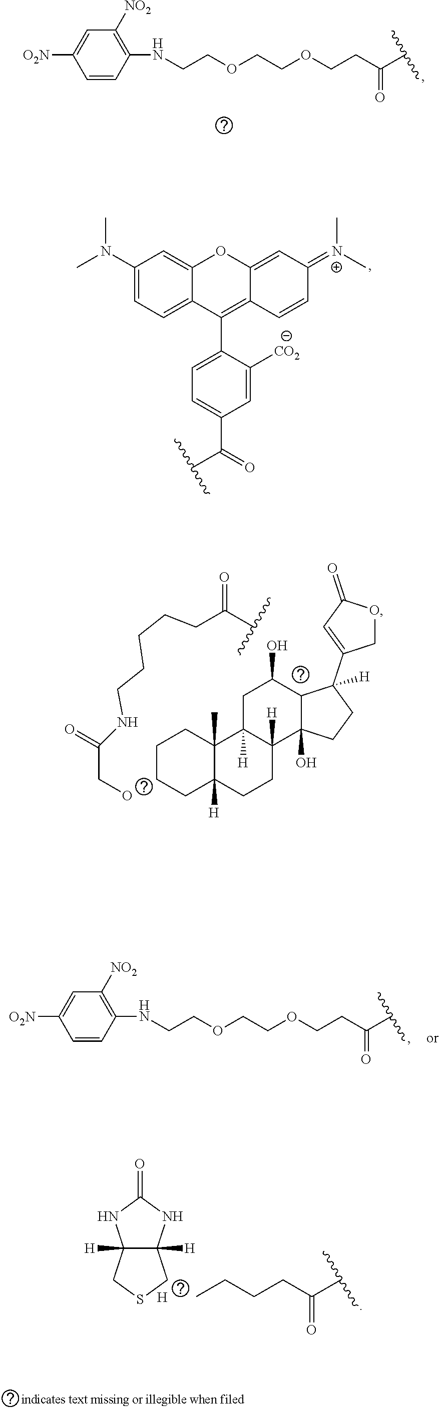

16. The CHT of claim 15, comprising formula II

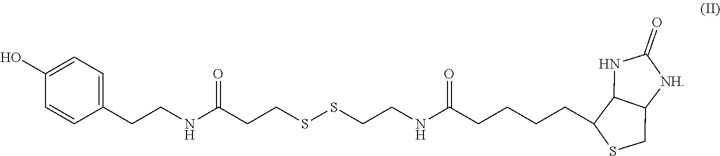 [SMILES: O=C(CCCCC1SCC2NC(=O)NC21)NCCSSCCC(=O)NCCc1ccc(O)cc1]

17. The method of claim 1, wherein before step a), the tissue is contacted with a quencher.

18. The method of claim 17, wherein the quencher is alkyne tyramide.

19. The method of claim 17, wherein before step f) the method further comprises performing a click chemistry reaction.
